SEQUENCE LISTING (22 sequences)

SEQ ID NO: 1 - protein, 601 aa, Artificial Sequence
  Description of Artificial Sequence scFv(UCHT-1)-PE38 amino acid sequence
MADIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRAGGGSGGGSGGGSGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSSKASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 2 - DNA, 1803 bp, Artificial Sequence
  Description of Artificial Sequence scFv(UCHT-1)-PE38 nucleotide sequence
atggcggacatccagatgacccagaccacctcctccctgtctgcctctctgggagacagagtcaccatcagttgcagggcaagtcaggacattagaaattatttaaactggtatcaacagaaaccagatggaactgttaaactcctgatctactacacatcaagattacactcaggagtcccatcaaagttcagtggcagtgggtctggaacagattattctctcaccattagcaacctggagcaagaggatattgccacttacttttgccaacagggtaatacgcttccgtggacgttcgctggaggcaccaagctggaaatcaaacgggctggaggcggtagtggcggtggatcgggtggaggcagcggtggcggatctgaggtgcagctccagcagtctggacctgagctggtgaagcctggagcttcaatgaagatatcctgcaaggcttctggttactcattcactggctacaccatgaactgggtgaagcagagtcatggaaagaaccttgagtggatgggacttattaatccttacaaaggtgttagtacctacaaccagaagttcaaggacaaggccacattaactgtagacaagtcatccagcacagcctacatggaactcctcagtctgacatctgaggactctgcagtctattactgtgcaagatcggggtactacggtgatagtgactggtacttcgatgtctggggcgcagggaccacggtcaccgtctcctcaaaagcttccggaggtcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacctgaag

SEQ ID NO: 3 - protein, 613 aa, Pseudomonas aeruginosa
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQTQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 4 - protein, 25 aa, Pseudomonas aeruginosa
MHLIPHWIPLVASLGLLAGGSSASA

SEQ ID NO: 5 - protein, 16 aa, Artificial Sequence
  Description of Artificial Sequence Linker
GGGSGGGSGGGSGGGS

SEQ ID NO: 6 - protein, 5 aa, Artificial Sequence
  Description of Artificial Sequence  PE peptide
REDLK

SEQ ID NO: 7 - protein, 4 aa, Artificial Sequence
  Description of Artificial Sequence PE peptide
REDL

SEQ ID NO: 8 - protein, 4 aa, Artificial Sequence
  Description of Artificial Sequence  PE peptide
KDEL

SEQ ID NO: 9 - protein, 5 aa, Artificial Sequence
  Description of Artificial Sequence peptide connector
KASGG

SEQ ID NO: 10 - protein, 5 aa, Artificial Sequence
  Description of Artificial Sequence peptide linker
GGGGS

SEQ ID NO: 11 - DNA, 32 bp, Artificial Sequence
  Description of Artificial Sequence  Primer IM-34A
gcggatccgacatccagatgacccagaccacc

SEQ ID NO: 12 - DNA, 32 bp, Artificial Sequence
  Description of Artificial Sequence  primer IM-34B
cctctagaagcccgtttgatttccagcttggt

SEQ ID NO: 13 - DNA, 35 bp, Artificial Sequence
  Description of Artificial Sequence  primer IM-34C
ccaagctttcatgaggagacggtgaccgtggtccc

SEQ ID NO: 14 - DNA, 29 bp, Artificial Sequence
  Description of Artificial Sequence  Primer IM-61
ccgtcgacgaggtgcagctccagcagtct

SEQ ID NO: 15 - DNA, 42 bp, Artificial Sequence
  Description of Artificial Sequence Oligo IM24A
ctagaggaggtagtggaggctcaggaggttctggaggtagtg

SEQ ID NO: 16 - DNA, 42 bp, Artificial Sequence
  Description of Artificial Sequence  Primer IM-24B
tcgacactacctccagaacctcctgagcctccactacctcct

SEQ ID NO: 17 - DNA, 23 bp, Artificial Sequence
  Description of Artificial Sequence  primer VL1
ctggtatcaacagaaaccagatc

SEQ ID NO: 18 - DNA, 27 bp, Artificial Sequence
  Description of Artificial Sequence  primer VL2
ggtgcctccagcgaacgtccacggaag

SEQ ID NO: 19 - DNA, 27 bp, Artificial Sequence
  Description of Artificial Sequence  Primer VL3
cttccgtggacgttcgctggaggcacc

SEQ ID NO: 20 - DNA, 21 bp, Artificial Sequence
  Description of Artificial Sequence  Primer VH4
ctctgcttcacccagttcatg

SEQ ID NO: 21 - DNA, 66 bp, Artificial Sequence
  Description of Artificial Sequence  Primer VL6
gccaccgctgcctccacctgatccaccgccactaccgcctccagcccgtttgatttccagcttggt

SEQ ID NO: 22 - DNA, 57 bp, Artificial Sequence
  Description of Artificial Sequence  Primer VL8
tcaggtccagactgctggagctgcacctcagatccgccaccgctgcctccacctgat

(19) United States
(12) Patent Application Publication
(10) Pub. No.: US 2002/0142000 A1
(43) Pub. Date: Oct. 3, 2002

(54) Anti-CD3 immunotoxins and therapeutic uses therefor

(76) Inventors: Mary Ellen Digan, Morristown, NJ (US); Philip Lake, Morris Plains, NJ (US); Richard Michael Wright, Annandale, NJ (US)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 09/480,236

(22) Filed: Jan. 10, 2000

(51) Int. Cl. A61K 39/395; A61K 39/40; A61K 39/42; A61K 39/44; C12P 21/08; C07K 16/00

(52) U.S. Cl. 424/183.1; 530/387.3; 530/388.75

(57) ABSTRACT

Recombinant immunotoxin polypeptides are described comprising a CD3-binding domain and a Pseudomonas exotoxin mutant, and in particular, comprising a single chain (sc) Fv as the CD3-binding moiety. A preferred species of the invention comprises scFv(UCHT-1)-PE38. Also disclosed are methods for the preparation of said immunotoxins; functionally equivalent immunotoxins which are intermediates in the preparation of the immunotoxins of the invention, as well as polynucleotide and oligonucleotide intermediates; methods for the prevention and/or treatment of transplant rejection and induction of tolerance, as well as treatment of autoimmune and other immune disorders, using the immunotoxins or pharmaceutically acceptable salts thereof; and pharmaceutical compositions comprising the immunotoxins or pharmaceutically acceptable salts thereof.

### FIELD OF THE INVENTION

The present invention relates to recombinant immunotoxins comprising a CD3-binding domain and a Pseudomonas exotoxin A mutant.

### BACKGROUND OF THE INVENTION

On the surface of every mature T cell are T-cell receptor (TCR) molecules consisting of a heterodimer of polypeptide chains α and β (or alternatively, chains γ and δ). The TCR α:β heterodimers, of which there are some 30,000 on every cell, are capable of engaging with the major histo-compatability complex (MHC) on an antigen-presenting cell (APC), and thereby account for antigen recognition by all functional classes of T cells. The α:β heterodimer itself does not appear to be involved in signal transduction following TCR engagement by specific MHC-peptide antigen complexes. Rather, that function is provided by a complex of proteins which is stably associated with the TCR αβ or γδ heterodimers on the surface of all peripheral T-cells and mature thymocytes, namely, the CD3 complex. The human CD3 complex comprises six polypeptides with usually four different chains: γ, δ, ε and ζ. Three different dimers constitute the CD3 complex (γε, δε, and ζζ), Leukocyte Typing VI, ed. by Kishimoto et al., Garland Publishing, Inc., 1998, p. 44. The CD3 proteins are absolutely essential for cell-surface expression of the T-cell receptor chains. Mutants lacking either of the TCR chains or any of the γ, δ or ε chains of the CD3 complex, fail to express any of the chains of the TCR at the cell surface. See Janeway, C. A., Jr. and P. Travers, Immunobiology. The Immune System in Health and Disease, Ch. 4 (“Antigen Recognition by T Lymphocytes”), Current Biology Ltd., London and Garland Publishing Inc., New York, 1996.

Antigen-specific T cell activation and clonal expansion occur when two signals are delivered by APC to the surface of resting T lymphocytes. The first signal, which confers specificity to the immune response, is mediated via the TCR following recognition of foreign antigenic peptide presented in the context of MHC. Optimal signaling through the TCR requires a clustering of the TCR with co-receptors CD4 or CD8. This in turn results in increased association of cytosolic tyrosine kinases with the TCR and the CD3 cytoplasmic tails, as well as with CD45. Phosphorylation of the cytoplasmic domain of CD3ε and ζ results in binding of tyrosine kinases, initiating a series of intracellular events resulting in the proliferation and differentiation of the T cell. The second signal, termed “costimulation,” which is neither antigen-specific nor MHC restricted, is provided by one or more distinct cell surface molecules expressed by APC's. Janeway and Travers, supra at 4-28.

Delivery of an antigen-specific signal with a costimulatory signal to a T cell leads to T cell activation, which can include both T cell proliferation and cytokine secretion. The combination of antigen and co-stimulator induces naïve T cells to express IL-2 and its receptor. IL-2 induces clonal expansion of the naïve T cell and the differentiation of its progeny into armed effector T cells that are able to synthesize all the proteins required for their specialized functions as helper, inflammatory, and cytotoxic T cells, see, e.g., Janeway and Travers, supra at §§7-8, 7-9.

The adaptive immune mechanisms described above constitute a major impediment to successful organ transplantation. When tissues containing nucleated cells are transplanted from a donor to a graft recipient, T-cell responses in the recipient to the typically highly polymorphic MHC molecules of the graft almost always trigger an immediate T-cell mediated response against the grafted organ. The use of potent immunosuppressives such as cyclosporin A and FK-506 to inhibit T cell activation has increased graft survival rates dramatically, but with certain disadvantages, including life-long dependence on the drug by the graft recipient.

Development of improved means of immunosuppression in patients receiving organ transplants, or suffering from T-cell mediated immune disease, has been a constant objective in the field of transplantation. A particular objective of workers in the art is development of a therapeutic agent capable of inducing donor-specific immunologic tolerance in a patient, and thereby freeing the patient from otherwise continuous dependence on immunosuppressives.

The term “immunological tolerance” refers to a state of unresponsiveness by the immune system of a patient subject to challenge with the antigen to which tolerance has been induced. In the transplant setting, in particular, it refers to the inhibition of the graft recipient's ability to mount an immune response which would otherwise occur in response to the introduction of non-self MHC antigen of the graft into the recipient. Induction of immunological tolerance can involve humoral, cellular, or both humoral and cellular mechanisms.

Systemic donor-specific immunological tolerance has been demonstrated in animal models as well as in humans through chimerism as a result of conditioning of the patient through total body irradiation or total lymphoid irradiation, prior to bone marrow transplantation with donor cells, Nikolic, B. and Sykes, M. (1997) Immunol. Res. 16: 217-228.

However, there remains a critical need for a conditioning regimen for allogeneic bone marrow transplantation that will result in stable mixed multilineage allogeneic chimerism and long-term donor-specific tolerance, in the absence of radiation. Hematologic abnormalities including thalassemia and sickle cell disease, autoimmune states, and several types of enzyme deficiency states, have previously been excluded from bone marrow transplantation strategies because of morbidity associated with conditioning to achieve fully allogeneic bone marrow reconstitution. Conditioning approaches which do not involve radiation may significantly expand the application of bone marrow transplantation for non-malignant diseases.

Immunotoxins comprising an antibody linked to a toxin have been proposed for the prophylaxis and/or treatment of organ transplant rejection and induction of immunological tolerance. For example, a chemically conjugated diphtheria immunotoxin directed against rhesus CD3ε, i.e. FN18-DT390, has been used in primate models of allograft tolerance and also in primate islet concordant xenograft models, see Knechtle et al.(1997) Transplantation 63:1, Neville et al. (1996) J. Immunother. 19: 85; Thomas et al. (1997) Transplantation 64: 124; Contreras et al. (1998) Transplantation 65: 1159-1169. Additionally, a chemically coupled Pseudomonas immunotoxin, LMB-1 B3(Lys)-PE38, has been used in clinical trials against advanced solid tumors, Pai, L. H. and I. Pastan, Curr. Top. Microbiol. Immunol. 234:83-96 (1998). However, product heterogeneity is a significant practical difficulty associated with chemically conjugated immunotoxins.

A single chain recombinant immunotoxin comprising the variable region of an anti-CD3 antibody, UCHT-1 and a diphtheria toxin, has been proposed as a therapeutic agent, see WO 96/32137, WO 98/39363. However, early vaccination of the general population against diphtheria raises concerns about pre-existing antibodies to the toxin in many patients. Alternately, a recombinant immunotoxin comprising anti-Tac linked to PE38 is also proposed as a prophylaxis and treatment against organ transplantation and autoimmune disease, see Mavroudis et al. (1996). Bone Marrow Transplant. 17: 793.

It has been an object to achieve a recombinant immunotoxin having directed toxic effect at high levels against T cells, which thereby provides improvements in the prophylaxis or treatment of transplant rejection and in the induction of immunologic tolerance, as well as in the treatment or prevention of graft versus host disease (GVHD), autoimmune disease, and other T-cell mediated diseases or conditions.

It has also been an object to provide an immunotoxin against which the recipient is normally free of pre-existing antibodies.

We have now discovered that recombinant fusions of a CD3-binding domain and a Pseudomonas exotoxin A mutant provide an immunotoxin having potent anti-T cell effect. The immunotoxins of the invention provide improvements in the clinical treatment or prevention of transplant rejection, graft-versus-host disease (GVHD), T-cell mediated autoimmune disease, T-cell leukemias, or lymphomas which carry the CD3 epitope, acquired immune deficiency syndrome (AIDS), and other T-cell mediated diseases and conditions.

### SUMMARY OF THE INVENTION

The present invention is directed to isolated recombinant immunotoxins comprising a CD3-binding domain and a Pseudomonas exotoxin A component, and pharmaceutically acceptable salts thereof; to in vivo and ex vivo methods for the treatment and prophylaxis of organ transplantation rejection and graft-versus-host disease, and for the induction of immunologic tolerance, as well as for treatment or prophylaxis of auto-immune diseases, AIDS and other T-cell mediated immunological disorders, and T-cell leukemias or lymphomas, using the immunotoxins or pharmaceutically acceptable salts thereof; and to pharmaceutical compositions comprising the novel immunotoxins or their pharmaceutically acceptable salts.

The invention also concerns polynucleotides and physiologically functional equivalent polypeptides which are intermediates in the preparation of the subject recombinant immunotoxins; recombinant expression vectors comprising said polynucleotides, procaryotic and eucaryotic expression systems, and processes for synthesizing the immunotoxins using said expression systems; and methods for purification of the immunotoxins of the invention.

In particular, the invention relates to a novel recombinant immunotoxin, scFv(UCHT-1)-PE38, which is a single chain (“sc”) Fv fragment of murine anti-human CD3 monoclonal antibody, UCHT-1, fused to a truncated fragment of Pseudomonas aeruginosa exotoxin A, i.e. PE38. For example, we have found said scFv(UCHT-1)-PE38 to be highly effective in T-cell killing in vitro; and we have further found that the immunotoxin is capable of ablating murine CD3/human CD3 double positive T cells at high levels in a dose-dependent manner in vivo in mice transgenic for human CD3ε.

### BRIEF DESCRIPTION OF THE FIGURES

FIG. 1 Schematic diagram showing domain organization of scFv(UCHT-1)-PE38 molecule prepared in Example 1, consisting of an N-terminal light chain variable region (V_{L}) of 109 residues, a peptide linker (L) of 16 residues, a heavy chain variable region (V_{H}) of 122 amino acids, a conector segment (C) of 5 amino acids (KASGG) (SEQ. ID. NO:9), and the PE38 mutant, comprising 347 amino acids (“Toxin”).

FIG. 2 Schematic map of pET15b expression plasmid prepared in Example 1 for expression of scFv(UCHT-1)-PE38 expression under control of bacteriophage T7 promoter (pT7) in E. coli. Relevant restriction sites, i.e. Nco I, Hind III and Bam HI/Bgl II, are noted. The peptide linker, (Gly₃Ser)₄ (SEQ. ID. NO: 5), is shown linking the carboxy terminus of V_{L} to the amino terminus of V_{H}.

FIG. 3 Typical elution profiles from anion-exchange columns used to purify scFv(UCHT-1)-PE38 in Example 1. (A) Step elution from Fast-Flow Q (Pharmacia). (B) Salt gradient elution from Q5 (BioRad).

FIG. 4 SDS-PAGE gel of scFv(UCHT-1)-PE38 (Lane 1: High molecular weight markers (Amersham); Lane 2: 2 μg refolded and concentrated protein prior to anion exchange column chromatography; Lane 3: 2 μg protein eluting at the peak position of the Fast Flow Q column; Lane 4: 2 μg protein eluting at the peak position from the Q5 column; Lane 5: High molecular weight markers (Amersham), including bovine serum albumin at 66 kD).

FIGS. 5A,B (A) Absorbance profile at OD₂₆₀ of scFv(UCHT-1)-PE38 on size exclusion chromatography (Sephacryl S200). (B) Mobility relative to the mobility of marker proteins (β-amylase, 200 kD; alcohol dehydrogenase, 150 kD; bovine serum albumin, 66 kD; carbonic anhydrase, 29 kD; cytochrome c, 12.4 kD).

FIG. 6 Protein synthesis in Jurkat (CD3⁺) compared to Ramos (CD3⁻) cells treated with increasing molar concentrations of scFv(UCHT-1)-PE38 (Pooled batches 12-16 and 10A-12A of Example 1), as a percent of protein synthesis in control, untreated cells of the respective type.

FIGS. 7A,B Inhibition of human mixed leukocyte reaction by scFv(UCHT-1)-PE38 or cyclosporine A (CsA) (positive control). As reported in Example 1, two different experiments, graphically represented in 7A and 7B, utilize cells from three different donors (A, B and C) in combinations A⇄B, A⇄C and B⇄C. ³H-TdR uptake by treated cells (relative to control, non-treated cells) is plotted against immunotoxin concentration (ng/ml) or CsA concentration (nM).

FIG. 8 Comparison of the effect of scFv(UCHT-1)-PE38 on proliferation of Con A-stimulated splenocytes from transgenic mice (“HuCD3εTg cells”) vs. cells from non-transgenic, B6CBAF1 mice (“NonTg cells”). ³H-thymidine incorporation (in counts per million, CPM) by the Conconavalin A (“ConA”)-stimulated T cells is plotted against scFv(UCHT-1)-PE38 concentration (ng/ml). Values represent the average of triplicate samples, and error bars represent the standard deviation. Solid horizontal lines represent the proliferative response in the absence of ConA, i.e. due to media alone: for the transgenic cells, this value is 342 cpm; for the nontransgenic cells this value is 112 cpm (not shown). In the transgenic cells, the value for a 50% proliferative response is 11,101 cpm. As reported in Example 1, the immunotoxin blocks ConA-induced proliferation of HuCD3εTg cells on a dose-dependent basis, but not of NonTg cells.

FIGS. 9A,B ³H-Thymidine incorporation (CPM) in one-way MLR. scFv(UCHT-1)PE38 (ng/ml) is shown to inhibit mitomycin C-induced proliferation of transgenic murine T cells expressing human CD3ε cells(“CD3Tg cells”) but not of non-transgenic, B6CBAF1 splenocytes (“NonTg cells”). Values represent the average of triplicate samples, and error bars represent the standard deviation. The line labelled “No stimulator cells” represents the proliferative response in the absence of Balb/C splenocytes, due to media alone (FIG. 9A: 1651 cpm; FIG. 9B: 342 cpm). In the transgenic cells, the value for a 50% proliferative response is 3891 cpm (FIG. 9A) or 688 cpm (FIG. 9B).

FIG. 10 Relative cell growth of CD3⁺ Jurkat cells, as compared to CD3⁻ LS174T and MDA-MB-435S cells, in hollow fibers implanted in the peritoneal cavity in nude mice (6 per group) administered scFv(UCHT-1)-PE38 by intraperitoneal injection (1 μg/mouse or 5 μg/mouse). Controls taken at Day 0 and on injection of vehicle alone are shown. Viable cell population is determined by MTS assay.

FIGS. 11A,B,C Two-color FACS analysis of spleen cells from heterozygous tgε600 transgenic mice with and without scFv(UCHT-1)-PE38 treatment. A. Non-specific double staining of spleen cells from untreated animals with isotype-matched control antibodies (“PE-Isotype” and “FITC-Isotype”). B. Double staining of spleen cells from untreated control animal with anti-mouse CD3-PE38 and anti-human CD3-FITC. C. Double staining with anti-mouse CD3-PE and anti-human CD3-FITC of spleen cells from an animal systemically treated with scFv(UCHT-1)-PE38 by intravenous injection.

FIGS. 12A,B,C Two-color FACS analysis of lymph node (LN) cells from heterozygous tgε600 transgenic mice with and without scFv(UCHT-1)-PE38 treatment. A. Double staining of LN cells from untreated animals with isotype control antibodies (PE-Isotype and FITC-Isotype). B. Double staining of lymph node cells from an untreated control animal with anti-mouse CD3-PE and anti-human CD3-FITC. C. Double staining with anti-mouse CD3-PE and anti-human CD3-FITC of LN cells from an animal systemically treated with scFv(UCHT-1)-PE38 by intravenous injection.

FIGS. 13A,B Decreasing fraction (A) and number (B) of transgenic human CD3-positive T spleen cells after systemic administration of scFv(UCHT-1)-PE38. The number of huCD3⁺ cells is determined by multiplying the total number of cells recovered from the spleen by the fraction of total cells (shown in FIG. 12A) that are huCD3⁺. (p<0.05 vs. untreated using one-way ANOVA of ranks).

FIGS. 14A,B Decreasing percentage (A) and number (B) of transgenic human CD3-positive lymph node (LN) cells after systemic administration of scFv(UCHT-1)-PE38. The number of huCD3⁺ cells is determined by multiplying the total number of cells recovered from the LN's by the fraction of total cells (shown in FIG. 14A) that are huCD3⁺. (p<0.05 vs. untreated using a one-way ANOVA of ranks).

FIG. 15 Nucleotide and amino acid sequence of scFv(UCHT-1)-PE38. DNA sequence encoding the NcoI, HindIII, EcoRI, and BamHI/BglII restriction sites used for subcloning, are underlined; the flexible linker separating the V_{L} from the V_{H} domains is also underlined. Numbers correspond to nucleotides. Single letter codes denote encoded amino acids. The amino-terminal residues Met and Ala are encoded by the NcoI restriction site that was added to facilitate expression from the E. coli plasmid pET 15b. The 3′ non-coding DNA between the EcoRI site and the BglII/BamHI site is carry-over sequence from the polylinker of an intermediate cloning vector (pLitmus 38, New England Biolabs).

FIGS. 16A-F Schematic depiction of certain immunotoxin constructs according to the invention.

### BRIEF DESCRIPTION OF THE SEQUENCE IDENTIFICATION NOS

SEQ. ID. NO:1 Amino acid sequence of scFv(UCHT-1)-PE38.

V_{L}=residues 3-111, linker=residues 112-127,

V_{H}=residues 128-249, connector plus truncated

PE=residues 250-601.

SEQ. ID. NO:2 Nucleotide sequence of scFv(UCHT-1)-PE38.

SEQ. ID. NO:3 Amino acid sequence of native Pseudomonas aeruginosa exotoxin A (mature protein).

SEQ. ID. NO:4 Signal sequence of Pseudomonas aeruginosa exotoxin A.

SEQ. ID. NO:5 Linker (Gly₃Ser)₄ of scFv(UCHT-1)-PE38.

SEQ. ID. NO:6 Carboxy terminus of PE (ArgGluAspLeuLys).

SEQ. ID. NO:7 Peptide sequence for PE (ArgGluAspLeu).

SEQ. ID. NO:8 Peptide sequence for PE (LysAspGluLeu).

SEQ. ID. NO:9 Connector peptide of scFv(UCHT-1)-PE38 (LysAlaSerGlyGly).

SEQ. ID. NO:10 Diabody linker (Gly₄Ser)

SEQ. ID. NOs: 11-22 Primers and oligos used in Example 1.

All oligopeptide and polypeptide formulas or sequences herein are written from left to right and in the direction from amino terminus to carboxy terminus.

### DETAILED DESCRIPTION OF THE INVENTION

1. CD3-Binding Domain.

The term “CD3-binding domain” refers to an amino acid sequence capable of binding or otherwise associating with mammalian, and more preferably primate, and even more preferably, human, CD3 antigen on T cells or lymphocytes.

The CD3-binding domain of the immunotoxins of the invention is preferably a polyclonal or monoclonal antibody to CD3, and more preferably, is a monoclonal anti-CD3 antibody. Even more preferably, the anti-CD3 antibody is a monoclonal antibody which is capable of binding an epitope on the ε chain of human CD3, or alternatively an epitope formed by the ε and γ chains of human CD3.

The term “antibody” as used herein includes intact immunoglobulins as well as various forms of modified or altered antibodies, including fragments of antibodies, such as an Fv fragment, an Fv fragment linked by a disulfide bond, or a Fab or (Fab)′₂ fragment, a single chain antibody, and other fragments which retain the antigen binding function and specificity of the parent antibody. The antibody may be of animal (especially, mouse or rat) or human origin or may be chimeric or humanized. Methods of producing antibodies capable of binding specifically to CD3 antigen, and more particularly, human CD3 antigen, may be produced by hybridomas prepared using well-known procedures deriving from the work of Kohler and Milstein, Nature, 256:495-97 (1975).

As is well-known in the art, an antibody “heavy” or “light” chain has an N-terminal variable region (V), and a C-terminal constant region (C). The variable region is the part of the molecule that binds to the antibody's cognate antigen, while the constant region determines the antibody's effector function.

Full length immunoglobulin or antibody heavy chains comprise a variable region of about 116 amino acids and a constant region of about 350 amino acids. Full-length immunoglobulin or antibody light chains comprise an N-terminal variable region of about 110 amino acids, and a constant region of about 110 amino acids at the COOH-terminus.

The heavy chain variable region is referred to as V_{H}, and the light chain variable region is referred to as V_{L}. Typically, the V_{L} will include the portion of the light chain encoded by the V_{L} and J₁ (i.e. joining region) gene segments (Sakans et al. (1979) Nature 280:288-294), and the “V_{H}” will include the portion of the heavy chain encoded by the V_{H}, D_{H} (i.e. diversity region) and J_{H} gene segments (Early et al. (1980) Cell 19:981-92).

The term “F(ab′)₂” used hereinabove refers to a divalent fragment of an antibody including the hinge regions and the variable and first constant regions of the heavy and light chains, which can be produced by pepsin digestion of the native antibody molecule, or by recombinant means. The term “Fab” refers to a monovalent fragment of an antibody including the variable and first constant regions of the heavy and light chains, which can be generated by reducing the disulfide bridges of the F(ab′)₂ fragment, or by recombinant means.

The V_{H} and V_{L} fragments together are referred to as “Fv”. The Fv region of an intact antibody is a heterodimer of (i.e. comprises on separate chains) the V_{H} and the V_{L} domains.

As is well-known in the art, an immunoglobulin light or heavy chain variable region comprises three hypervariable regions, also called complementarity determining regions (CDR's), flanked by four relatively conserved “framework regions” (FR's).

The combined framework regions of the constituent light and heavy chains serve to position and align the CDR's. The CDR's are primarily responsible for binding to an epitope of an antigen and are typically referred to as CDR1, CDR2 and CDR3, numbered sequentially starting from the N-terminus of the variable region chain. Framework regions are similarly numbered.

Numerous framework regions and CDR's have been described (see, “Sequences of Proteins of Immunological Interest,” E. Kabat and Wu, U.S. Government Printing Office, NIH Publication No. 91-3242 (1991) (“Kabat and Wu”). The CDR and FR polypeptide segments are designated empirically based on sequence analysis of the Fv region of preexisting antibodies or of the DNA encoding them. From alignment of antibody sequences of interest with those published in Kabat and Wu and elsewhere, framework regions and CDRs can be determined for the antibody or other CD3 binding region of interest.

By “chimeric” is generally meant a genetically engineered antibody comprising sequences derived from more than one natural antibody. An example of a chimeric antibody is one in which the framework and complementarity determining regions are from different sources, as when a non-human variable domain is linked to a human constant domain. As a subset thereof, a “humanized” antibody is generally understood to comprise an antibody wherein non-human CDRs are integrated into framework regions at least a portion of which are human.

As used herein, the term “single chain antibody” (or the term “single chain immunotoxin”) refers to a molecule wherein the CD3-binding domain is on a single polypeptide chain.

Single chain antibodies are typically prepared by determining and isolating the binding domain of each of the heavy and light chains of a binding antibody, and supplying a linking moiety which permits preservation of the binding function. This forms, in essence, a radically abbreviated antibody, having, on a single polypeptide chain, only that part of the variable domain necessary for binding to the antigen. Methods for preparation of single chain antibodies are described by Ladner et al., U.S. Pat. No. 4,946,778, incorporated by reference.

A single chain immunotoxin according to the invention comprises such a single chain antibody fragment. The toxin component is preferably fused to the CD3-binding domain(s), optionally via a linker peptide, but may also exist as a separate polypeptide chain linked via one or more disulfide bonds to the chain containing the CD3-binding domain.

An immunotoxin of the invention may be “monovalent,” by which is meant that it contains one CD3-binding domain (e.g., the combined V_{H} and V_{L} variable regions of an antibody) on the chain.

An immunotoxin of the invention may also be “divalent,” by which is meant that it contains two CD3-binding domains. The two antigen-binding domains can be located on a single chain, or alternatively, on two or more chains linked by disulfide bonds or otherwise in close association due to attractive forces (e.g., hydrogen bonds). When two CD3-binding domains are on a single chain, they may be present in tandem (i.e. following consecutively in series in the chain, bound together by a peptide bond or linker), or else separated on the chain by an intervening PE mutant, or other functional domains.

Single chain antibodies (or single chain immunotoxins) may multimerize upon expression, depending on the expression system, by formation of interchain disulfide bonds with other single (or double) chain molecules, or by means of the intrinsic affinity of domains for their partner. The chains can form homodimers or heterodimers.

The CD3-binding moiety of the immunotoxins of the invention is preferably a “recombinant” antibody. Likewise, the immunotoxins of the invention are “recombinant” immunotoxins. By the use of the term “recombinant” it is understood that the antibody (or immunotoxin) is synthesized in a cell from nucleotide (e.g., DNA) segments produced by genetic engineering. The term “isolated” indicates that a polypeptide has been removed from its native environment. A polypeptide produced and/or contained within a recombinant host cell is considered isolated for purposes of the present invention. Also intended as an “isolated polypeptide” are polypeptides that have been purified, partially or substantially, from a recombinant host cell.

Preferably, the CD3-binding moiety of the immunotoxins of the invention is a single chain (“sc”) antibody. The immunotoxin is preferably monovalent.

Most preferably, the CD3-binding moiety of the invention comprises a single chain Fv region (or CD3-binding fragment thereof) of an antibody, i.e. wherein the V_{H} region (or CD3-binding portion thereof) is fused to the V_{L} region (or CD3-binding portion thereof), optionally via a linker peptide.

The V_{L} region is preferably linked via its carboxy terminus to the amino terminus of the V_{H} region; alternatively, the V_{H} region may be linked via its carboxy terminus to the amino terminus of the V_{L} region.

Any peptide linker of the V_{L} and V_{H} regions preferably allows independent folding and activity of the CD3-binding domain; is free of a propensity for developing an ordered secondary structure which could interfere with the CD3-binding domain or cause immunologic-reaction in the patient, and has minimal hydrophobic or charged characteristic which could interact with the CD3-binding domain.

The peptide connector is preferably 1-500 amino acids; more preferably 1-250; and even more preferably no more than 1-100 (e.g., about 1-25 or 10-20) amino acids.

For each of the above preferences, the linker is preferably linear.

In general, linkers comprising Gly, Ala and Ser can be expected to satisfy the criteria for such a peptide.

For example, the linker in scFv(UCHT-1)-PE38, linking the carboxy terminus of the V_{L} domain to the amino terminus of the V_{H} domain, is [(Gly₃)Ser]₄ (SEQ. ID. NO: 5).

Examples of specific anti-CD3 antibodies the whole or fragments of which are suitable to be employed as a CD3-binding domain of the invention are:

(1) UCHT-1 (Beverley P. C. L. and Callard, R. E. (1981) Eur. J. Immunol. 11: 329; and Burns, G. F. et al. (1982) J. Immunol. 129: 1451), the scFv sequence of which is included in SEQUENCE ID NO:1. UCHT-1 is a monoclonal mouse anti-human anti-CD3 antibody having an IgG1, Kappa isotype. The antibody reacts with T cells in thymus, bone marrow, peripheral lymphoid tissue, and blood. The intact antibody is commercially available from Biomeda (Catalog No. K009, V1035) or Coulter Corp. The variable regions comprise residues 3 to 112 (light chain) and 128 to 249 (heavy chain) of SEQ. ID NO:1 herein. UCHT-1 is non-activating as an Fv fragment and has been used as a fusion partner with anti-HER2 bispecific immunoconjugates in targeting T-cells to human breast and ovarian tumor cells (see Shalaby et al. (1992), J. Exp. Med. 175:217).

(2) SP34 (first isolated by C. Terhorst, Beth Israel Deaconess Hospital), reacts with both primate and human CD3. SP34 differs from UCHT-1 and BC-3 (described below) in that SP-34 recognizes an epitope present on solely the ε chain of CD3 (see Salmeron et al., (1991) J. Immunol. 147: 3047) whereas UCHT-1 and BC-3 recognize an epitope contributed by both the ε and γ chains. The intact antibody is commercially available from PharMingen.

(3) BC-3 (Fred Hutchinson Cancer Research Institute) (used in Phase I/II trials of GvHD) (Anasetti, et al., (1992) Transplantation 54: 844).

Other monoclonal antibodies having specific binding affinity for CD3 antigen and having at least some sequences of human origin are considered to be within the scope of homologs of the abovementioned antibodies. These antibodies include: (1) a monoclonal antibody having complementarity-determining regions identical with, for example, UCHT-1 (or SP34 or BC3) and having at least one sequence segment of at least five amino acids of human origin; and (2) a monoclonal antibody competing with, e.g., UCHT-1, for binding to human CD3 antigen at least about 80%, and more preferably at least about 90%, as effectively on a molar basis as UCHT-1, and having at least one sequence segment of at least five amino acids of human origin. By “specific binding affinity” is meant binding affinity determined by noncovalent interactions such as hydrophobic bonds, salt linkages, and hydrogen bonds on the surface of binding molecules. Unless stated otherwise, “specific binding affinity” implies an association constant of at least about 10⁶ liters/mole for a bimolecular reaction.

Antibodies of this invention having complementarity-determining regions substantially homologous with those of, e.g., UCHT-1, are also within the scope of this invention and can be generated by in vitro mutagenesis. Among the mutations that can be introduced into constant or variable regions that substantially preserve affinity and specificity of such homologs are mutations resulting in conservative amino acid substitutions, such as are well-known in the art. With respect to UCHT-1, such mutant forms of antibodies preferably have variable regions which are at least 80% identical, and more preferably at least 90% identical, to the variable region of UCHT-1. Even more preferably, each of the complementarity-determining regions of such mutant forms of antibodies is at least 80%, and more preferably at least 90%, or at least 95%, identical to the corresponding complementarity-determining region of UCHT-1.

As a practical matter, whether any particular polypeptide sequence is at least 80%, 90%, or at least 95%, “identical to” another polypeptide can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

The CD3 binding moiety of the invention in a preferred embodiment recognizes an epitope of human CD3 formed by both the γ and ε chains, and is preferably UCHT-1, and more preferably, is the Fv region (or CD3-binding fragment thereof) of UCHT-1.

Even more preferably, the CD3 binding moiety is a single chain fragment of UCHT-1, and most preferably, is a single chain Fv region (or CD3-binding fragment thereof) of UCHT-1.

It has been found that the Fv region of UCHT-1, when reconstituted as a single chain and fused to a cell-binding domain-deleted fragment of Pseudomonas aeruginosa exotoxin A, demonstrates high levels of potency in T-cell killing in standard in vitro assays and in vivo in transgenic mice heterozygous for human CD3ε.

2. Pseudomonas Toxin Component.

Pseudomonas exotoxin-A (hereinafter, “PE”) is an extremely active monomeric protein of 613 amino acids (molecular weight 66 Kd), secreted by Pseudomonas aeruginosa, which inhibits protein synthesis in eukaryotic cells through inactivation of elongation factor 2 (EF-2), an essential eukaryotic translation factor by catalyzing its ADP-ribosylation (i.e. catalyzing the transfer of the ADP ribosyl moiety of oxidized NAD onto EF-2), see Kreitman and Pastan (1994) Blood 83: 426.

The mature polypeptide has the amino acid sequence set forth in SEQ. ID NO:3 herein, which normally is preceded by a signal sequence of 25 residues as set forth in SEQ. ID NO:4.

Three structurally distinct domains in native PE act in concert to promote cytoxicity (see Pastan et al., U.S. Pat. No. 4,892,827, incorporated by reference; see also U.S. Pat. Nos. 5,696,237 and 5,863,745, also incorporated by reference). Domain Ia, at the amino terminus (and generally assigned residues 1 to about 252 of SEQ. ID NO:3), mediates cell targeting and binding. Domain II (at residues 253-364 of SEQ. ID NO:3) is responsible for translocation across the cell membrane into the cytosol; and Domain III (residues 405 to 613 of SEQ. ID NO:3) mediates ADP ribosylation of elongation factor 2, thereby inactivating the protein and causing cell death. Domain III contains a carboxy-terminal sequence (REDLK) (SEQ. ID. NO:6) that directs the endocytosed and processed toxin into the endoplasmic reticulum. While Domain Ib (residues 365-404 of SEQ. ID NO:3) appears to act in concert with Domain III, deletion of residues 365-380 of this domain results in no loss of activity.

The “PE mutant” or, alternatively “PE component,” of the immunotoxins of the invention is a mutant form of native PE having translocation and catalytic (i.e. ADP-ribosylating) functions but having substantially diminished or deleted cell-binding capability.

Disruption or deletion of all or substantially all of cell-binding Domain Ia has been found to substantially reduce the cell-binding capability and thus the non-specific toxicity of the native PE molecule.

For example, deletion of Domain Ia yields a 40 kDa protein, PE40, which itself is not cytotoxic despite retaining the translocation and ADP-ribosylation functions of domains II and III, respectively (Kondo et al., 1988, J. Biol. Chem, 263:9470-9475).

PE38 is a 38 kDa fragment of PE also essentially lacking Domain Ia of the mature PE protein (e.g., lacking amino acids 1-250 of SEQ. ID. NO: 3), and also lacking amino acid residues 365 to 380 of SEQ. ID. NO:3, and thus having the amino acid sequence comprising residues 251 to 364 joined to 381 to 613 of SEQ. ID NO:3 (see residues 255-601 of SEQ. ID. NO:1). See also U.S. Pat. No. 5,608,039, col. 10, 11. 1-20, where PE38 is indicated to refer to a truncated toxin composed of amino acids 253-364 and 381-613 of native PE. Advantageously, PE38 lacks the cysteine residues at positions 372 and 379 of the native protein, which otherwise can potentially form disulfide bonds with other cysteines during the renaturation process and can lead to formation of inactive chimeric toxins.

A PE toxin component of the polypeptides of the invention may also comprise a polypeptide which is at least 90% identical to, and more preferably at least 95% identical to, and even more preferably at least 99% identical to, the sequence defined by residues 255-601 of SEQ. ID. NO:1, wherein the term “identical to” has the significance indicated previously.

PE38KDEL has the amino acid sequence of PE38, described above, with the exception that the carboxyl terminus of the toxin is changed from the original sequence REDLK (SEQ. ID. NO: 6) to KDEL (SEQ. ID. NO: 8).

Other deletions or changes may be made in PE or in addition of a linker such as an IgG constant region connecting an antibody to PE, in order to increase cytotoxicity of the fusion protein toward target cells, or to decrease nonspecific cytotoxicity toward cells lacking the corresponding CD3 antigen. Deleting a portion of the amino terminal end of PE domain II increases cytotoxic activity, in comparison to the use of native PE molecules or those where no significant deletion of domain II has occurred. Other modifications include an appropriate carboxyl terminal sequence to the recombinant PE molecule to help translocate the molecule into the cytosol of target cells. Amino acid sequences which have been found to be effective include REDLK (SEQ. ID. NO: 6)(as in native PE), REDL (SEQ. ID. NO:7) or KDEL (SEQ. ID. NO:8) (as in PE38KDEL discussed above), repeats of those, or other sequences that function to maintain or recycle proteins into the endoplasmic reticulum, see Pastan, U.S. Pat. No. 5,489,525, incorporated by reference. Other mutants may comprise single amino acid substitutions (e.g., replacing Lys with Gln at positions 590 and 606).

Additional PE mutants having recognition moieties inserted into Domain III of PE are described by Pastan et al., U.S. Pat. No. 5,458,878, incorporated by reference.

3. Construction of Immunotoxins.

This invention includes fusions of a CD3-binding domain to one or more Pseudomonas mutants; and also includes immunotoxin fusions comprising two or more CD3-binding domains and at least one PE mutant.

The term “fused” or “fusion” as employed herein refers to polypeptides in which:

(i) a “first polypeptide domain” is bound at its carboxy terminus via a chemical (i.e. peptide) bond to the amino terminus of a “second polypeptide domain,” optionally via a peptide connector, or, conversely, where

(ii) the “second polypeptide domain” of (i) is bound at its carboxy terminus via a chemical (i.e. peptide) bond to the amino terminus of the “first polypeptide domain” of (i), optionally via a peptide connector.

Similarly, “fused” when used in connection with the polynucleotide intermediates of the invention means that the 3′-[or, conversely, 5′-] terminus of a nucleotide sequence encoding a first functional domain is bound to the respective 5′-[or conversely, 3′-] terminus of a nucleotide sequence encoding a second functional domain, either directly via a chemical (i.e. covalent) bond or indirectly via a connector nucleotide sequence which itself is chemically (i.e. covalently) bound to the first functional domain-encoding nucleotide sequence and the second functional domain-encoding nucleotide sequence via their termini.

Additional peptide sequences making up the fusions may be selected from full length or truncated (e.g., soluble, extracellular fragments of) human proteins. Examples of such peptide sequences include human immunoglobulin protein domains, domains from other human serum proteins, or other domains which can be multimerized (see Kostelny et al., 1992, J. Immunol. 148: 1547-1553; Tso et al., WO 93/11162; Pack and Pluckthun, 1992, Biochemistry 31: 1579-1584; Hu et al., 1996, Can. Res. 56: 3055-3061; Wu, WO 94/09817); Pack et al., 1995, J. Mol. Biol. 246: 28-34.

Said additional functional domains may also serve as peptide connectors, for example, joining the CD3 antigen-binding domain to the PE component; or alternatively, said additional domain(s) may be located elsewhere in the fusion molecule, e.g., at the amino or carboxy terminus thereof.

In a preferred embodiment of the invention, a single chain Fv of an anti-CD3 antibody is fused to a truncated fragment of PE having translocation and catalytic functions but substantially lacking cell binding capability.

Preferably, the antibody binding regions which recognize the CD3 antigen may be inserted in replacement for deleted domain Ia of the PE molecule. Thus in the various embodiments of the invention, it is preferred that the CD3-binding moiety be linked via its carboxy terminus (optionally through a connector peptide or other functional domain) to the amino terminus of the PE toxin component.

Alternatively, the PE toxin component may be linked via its carboxy terminus to the amino terminus of the CD3-binding moiety (also, optionally, via a connector peptide or other functional domain).

Where there are multiple CD3-binding domains on a single chain, these may be linked in tandem by a peptide bond or linker, or else separated by an intervening PE component or another functional moiety.

Any peptide connector linking the CD3-binding region and the PE component preferably allows independent folding and activity of the CD3-binding domain; is free of a propensity for developing an ordered secondary structure which could interfere with the CD3-binding domain or cause immunologic-reaction in the patient, and has minimal hydrophobic or charged characteristic which could interact with the CD3-binding domain.

The connector is preferably 1-500 amino acids; more preferably 1-250; and even more preferably no more than 1-100 (e.g., 1-25, 1-10, 1-7 or 1-4) amino acids.

For each of the above preferences, the connector is preferably linear.

In general, conector peptides linking the CD3-binding domain and the PE component which comprise small, uncharged amino acids can be expected to satisfy the criteria for such a connector. For example, the connector peptide in sc(UCHT-1)-PE38 is Lys-Ala-Ser-Gly-Gly (KASGG) (SEQ. ID. NO:9). Other peptides of various lengths and sequence composition may also be useful.

Most preferably, the immunotoxin of the invention is a single chain polypeptide comprising the Fv region (or CD3-binding fragment thereof) of UCHT-1 fused via its carboxy terminus, optionally via a connector peptide, to the amino terminus of PE38.

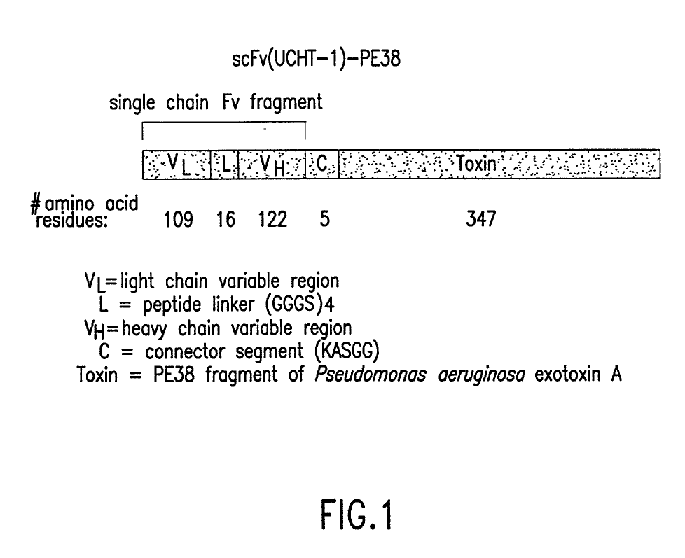

A schematic drawing of such a molecule is shown in FIG. 1. scFv(UCHT-1)-PE38 is a protein of 600 amino acids, having a predicted molecular weight of 64,563 daltons (64.5 kD).

It will be noted that the actual translation product from E. coli of the molecule schematically depicted in FIG. 1 may comprise an added N-terminal methionine (Met) residue, because of incomplete cleavage of the Met normally supplied to a coding sequence to initiate transcription from E. coli. Additionally, the scFv(UCHT-1)PE38 polypeptide prepared according to Example 1 may contain an added alanine (Ala) at the N-terminus or at position 2 (i.e. following Met) as a result of sequence added at the N-terminus to facilitate cloning. The mature amino terminus of the variable region of the light chain of UCHT-1 begins at position 3 of SEQ. ID. NO:1, i.e. aspartic acid (Asp). Accordingly, E. coli expression of the molecule depicted in FIG. 1 as prepared according to Example 1 may yield one or more of the following functionally equivalent products, depending on the expression strain used, and the precise fermentation and purification conditions used: the polypeptide having sequence 1-601 of SEQ. ID. NO:1 and encoded by nucleotides 1-1803 of SEQ. ID. NO: 2; the polypeptide having sequence 2-601 of SEQ. ID. NO:1 and encoded by nucleotides 4-1803 of SEQ. ID. NO:2; and the polypeptide having sequence 3-601 of SEQ. ID. NO:1 and encoded by nucleotides 7-1803 of SEQ. ID. NO:2.

It shall be understood that any of such forms of the protein (or the corresponding nucleic acid) are encompassed by the term “scFv(UCHT-1)-PE38” as employed herein, unless otherwise indicated.

This invention also encompasses polypeptides which are at least 80% identical to, and more preferably at least 90% identical to, and even more preferably, at least 95% identical to, the polypeptide having SEQ. ID. NO:1, wherein the term “identical to” has the meaning previously indicated.

Certain immunotoxin molecules may be “dimerized” by the attractive forces between domains located on the polypeptide chains or by the formation of disulfide bonds between cysteine residues.

For example, a dimer may be formed from two polypeptide chains, or from two pairs of chains. Dimers may be homodimers or heterodimers (An example of a hetereodimer is a construct in which the PE toxin is present on only one of two chains.)

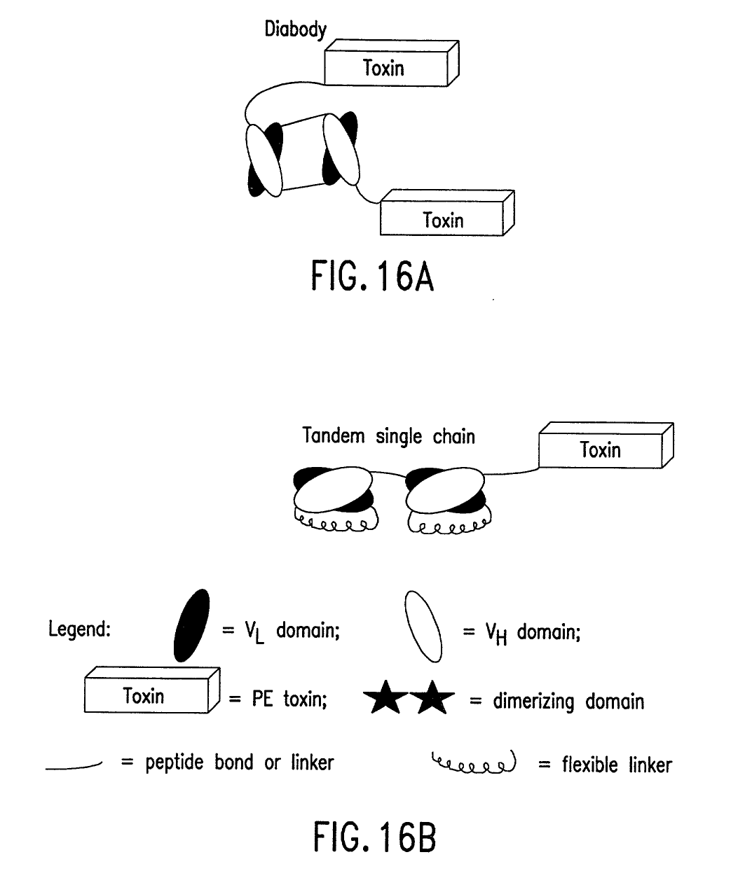

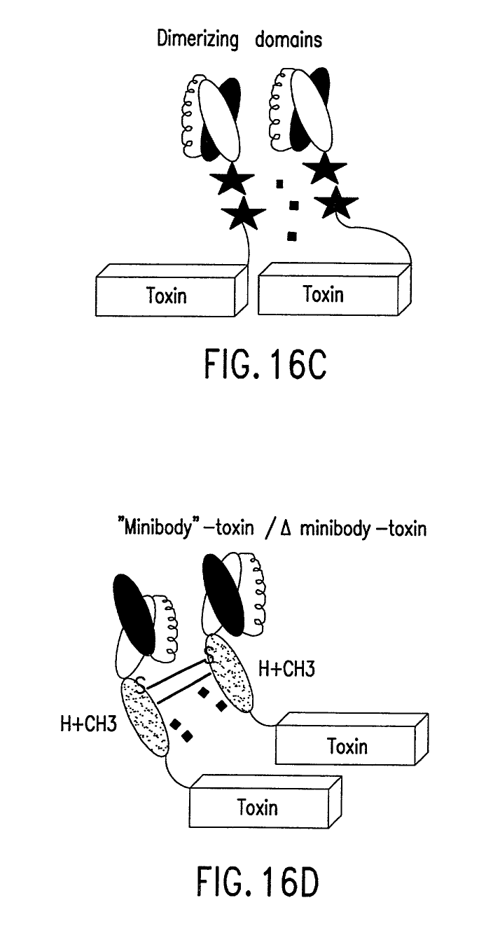

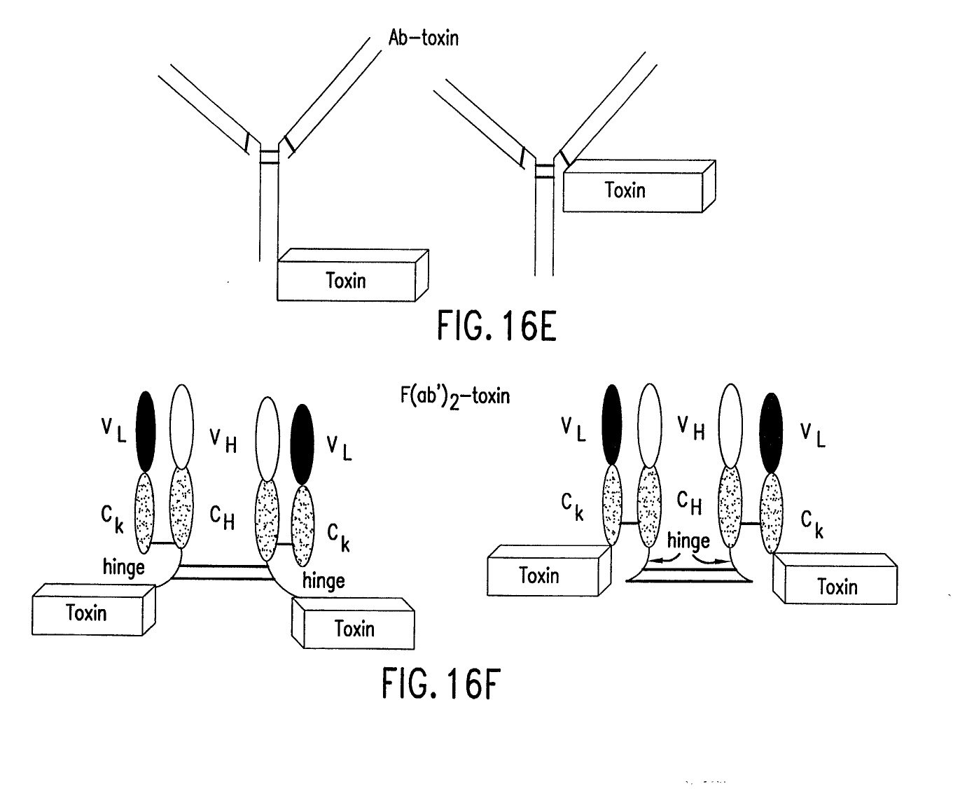

Certain divalent single chain immunotoxin constructs, or dimerized constructs, according to the invention are illustrated in FIG. 16.

The dimerized immunotoxin constructs depicted in FIGS. 16A, C, D, E and F comprise two (or more) chains. The construct depicted in FIG. 16B is a divalent single chain immunotoxin. The molecules shown in FIG. 16E are full length recombinantly prepared antibodies linked to a toxin. The construct of FIG. 16F is a recombinantly prepared F(ab′)₂ fragment (i.e. comprising a dimer of two pairs of chains) linked to toxin.

The PE toxin in the constructs depicted in FIG. 16 is preferably PE38, and the antibody variable domains may be derived from UCHT-1.

In particular, a first illustrative embodiment of a dimeric immunotoxin of the invention is a diabody, as illustrated in FIG. 16A.

By “diabody” is meant an immunotoxin construct comprising two (preferably identical) single chains, each chain comprising V_{L} and V_{H} domains and a PE mutant toxin, said chains becoming associated due to attractive forces between the variable domains (e.g., hydrogen bonding, not represented in FIG. 16A) rather than by disulfide bonding.

FIG. 16A depicts a pair of single chains having the configuration, V_{L}-L-V_{H}-PE mutant toxin, as shown.

By contrast with the single chain immunotoxin schematically diagrammed in FIG. 1, for purposes of preventing intrachain Fv formation, the linker L between the V_{L} and V_{H} domains in each polypeptide chain of a diabody is preferably substantially inflexible, and is generally no greater than 10 amino acids, and is more preferably no greater than 1-5 amino acids, as exemplified by the linker: (Gly)₄Ser (SEQ. ID. NO:10), and can even be absent entirely. (In contrast, the linker between V_{L} and V_{H} in a single chain immunotoxin is preferably at least about 14 amino acids.) Thus the functional Fv region of a diabody is actually formed by the interaction of the two chains together. Diabodies may be expressed from mammalian cells as well as E. coli.

Diabody construction has been described in general by Hollinger et al., (1993) Proc. Nat. Acad. Sci. 90: 6444, and Wu et al. (1996) Immunotech 2:21.

In another illustrative embodiment of the invention, a tandem single chain construct, as depicted in FIG. 16B, comprises two anti-CD3 Fv regions consecutively linked in series, i.e. by a peptide bond or via a peptide linker which is optionally flexible.

FIG. 16B depicts a construct having the configuration: V_{L}-L-V_{H}-X-V_{L}-L-V_{H}-Y-Toxin, wherein X and Y are independently selected from a peptide bond or linker. In particular, L may be a linker such as that depicted in FIG. 1 hereof, i.e. (GGGS)₄ (SEQ. ID. NO:5), and each of X and Y may have a sequence such as that of the “connector” also described in FIG. 1 (i.e. KASGG, SEQ. ID. NO:9).

Similar to the construct shown in FIG. 1, the V_{L} and V_{H} domains of each of the two Fv regions are separated by a peptide linker L which is flexible (represented in FIG. 16B, as well as in FIGS. 16C and D, by a looping line connecting each V_{L} and V_{H} domain), having preferably about 10-30, and more preferably about 14 to 25, amino acids.

Preferably, the two Fv regions in the construct shown in FIG. 16B are both anti-CD3 binding domains. Thus in one embodiment, the Fv regions may bind to the same epitope of CD3, and may even be identical (or each region or its encoding nucleotide sequence may be modified to facilitate expression or inhibit recombination); or alternatively, each Fv may be selected to bind to a different epitope on human CD3 antigen.

A PE toxin component of the invention may be linked (optionally through intervening linkers or functional sequences) to the carboxy or the amino terminus of one of the Fv domains. (Alternatively, multiple PE toxin segments may be present in the molecule.) In FIG. 16B, the PE sequence is linked to the carboxy terminus of one of the Fv domains.

Tandem single chain antibody molecules in which the antigen binding regions bind to different antigens, rendering such molecules “bispecific”, are described in general by Gruber et al. (1994) J. Immunol. 152: 5368, Kurcucz and Segal (1995) J. Immunol. 154: 4576, Mallender et al., (1994) J. Biol. Chem. 269: 199, and Mack et al. (1995) Proc. Nat. Acad. Sci. 92: 7021.

Still another construct of the invention is prepared from two polypeptide chains each comprising a “dimerizing domain” which serves to facilitate dimerization between the chains by associational forces (e.g., hydrogen bonding), rather than by disulfide bonding. (The mentioned associational forces are represented by the dots in FIG. 16C, as well as in FIG. 16D.) Each dimerizing domain, depicted in FIG. 16C by a pair of stars, can be located internally within the chain, for example, between the Fv region and the PE toxin component (as shown); or in another aspect, the dimerizing domain may be located at the N-terminus of the Fv region (not shown); and in still another aspect, the dimerizing domain may be located at the C-terminus of the PE toxin (not shown). In the construct depicted in FIG. 16C, each chain has the configuration: V_{L}-L-V_{H}-dimerizing domain-PE mutant toxin.

Dimerizing domains are described in general by Pack and Pluckthun (1992) Biochem. 31: 1579 and Kostelny et al., supra. Suitable dimerizing domains may be derived from heterodimeric transcription factors or amphiphilic helices, and expressed in mammalian cells as well as E. coli.

Another dimerized construct according to the invention is prepared from single chain immunotoxins comprising the hinge and third constant region (“CH3”) of Ig to effect dimerization through formation of disulfide bonds and attractive forces between the CH3 segments.

As shown in FIG. 16D, a “minibody”-toxin of the invention may comprise two (e.g., identical) single chains, each of which chains comprises an Fv region linked via hinge (“H”) and CH3 of, e.g., human IgG1, to the PE toxin component. Each of the lightly shaded ovals in FIG. 16D represents the hinge and CH3 domains. Thus each chain has the configuration: V_{L}-L-V_{H}-H+CH3-PE mutant toxin. The polypeptide chains are linked by disulfide bonds (represented in FIG. 16D, as well as in FIGS. 16E and F, by thickened lines) as well as associational forces (represented by dots), between the respective hinge and CH3 domains. (A variant construct referred to in FIG. 16D as “Δminibody-toxin” is mutated to prevent mispairing of cysteines by replacing the cysteine in the hinge region which ordinarily pairs the heavy and light chains of the native antibody, with, e.g., serine or alanine, and leaving intact the two remaining cysteines in the hinge which bind the heavy chains.)

Other variants utilize the hinge from other immunoglobulin isotypes or other mammalian species, e.g., murine IgG's. A “minibody” has been described in general by Hu et al. (1996) Can. Res. 56: 3055.

Another illustrative construct according to the invention comprises a recombinant antibody fused via the C-terminus of either the heavy chain (FIG. 16E, left panel) or the light chain (FIG. 16E, right panel) to a PE mutant toxin according to the invention. As in the native antibody, the chains are linked by disulfide bonds (thickened lines connecting chains), as shown. Said full length antibody toxins generally dimerize in pairs. In such constructs, a non-huFcγ-receptor binding Ig, such as murine IGg2b or human IgG₄, may be substituted for the native Fc. Optionally, a PE toxin component may be present on both heavy and light chains (not shown).

An additional construct according to the invention comprises a recombinantly prepared F(ab′)₂ fragment (including the indicated hinge region), which is linked via the carboxy terminus of the heavy chain (FIG. 16F, left panel) or light chain (FIG. 16F, right panel)(optionally via a linker, not shown), to a PE mutant toxin. Said F(ab′)₂ toxin molecules generally dimerize in pairs. (The lightly shaded ovals in FIG. 16F represent either the constant domain of the heavy chain (“C_{H}” ) or the constant domain of the light chain (“C_{κ}”), as indicated. The hinge regions of the polypeptide chain are separately represented from the constant regions by the disulfide-linked connectors labelled “hinge”. Thus, the respective chains have the configuration V_{L}-C_{κ}and V_{H}-C_{H1}-hinge-PE toxin (FIG. 16F, left) or, alternatively, V_{L}-C_{κ}-PE toxin and V_{H}-C_{H1}-hinge (FIG. 16F, right).

The above constructs can be prepared from known starting materials by techniques of recombinant engineering known by workers skilled in the art.

The invention is also intended to include polypeptide homologs (and the DNA molecules which encode said polypeptides) which differ from a disclosed species of polypeptide by having, for example, conservative substitutions in amino acid over the disclosed polypeptide, or minor deletions or additions of residues not otherwise substantially affecting the CD3-binding ability or catalytic activity of the immunotoxin.

By “conservative substitution” is meant the substitution of one or more amino acids by others having similar properties such that one skilled in the art of polypeptide chemistry would expect at least the secondary structure, and preferably the tertiary structure of the polypeptide to be substantially unchanged. Conservative replacements are generally those that take place within a family of amino acids that are related in their side chains. Typical amino acid replacements include alanine or valine for glycine, asparagine for glutamine, serine for threonine and arginine for lysine.

Also within the scope of this invention are homologs of the species of immunotoxin disclosed herein.

The term “homolog” or “homology” refers to sequence similarity between two peptides or between two nucleic acid molecules. Homology can be determined by comparing a position in each sequence which may be aligned for purposes of comparison. When a position in the compared sequence is occupied by the identical base or amino acid, then the molecules are homologous at that position. A degree of homology between sequences is a function of the number of matching or homologous positions shared by the sequences.

Preferably, any homolog of an immunotoxin polypeptide species of the invention is at least 80% identical to, and preferably at least 90% identical to, and more preferably at least 95% identical to, said immunotoxin polypeptide of the invention.

All of the amino acids of the polypeptides of the invention (except for glycine) are preferably naturally-occurring L-amino acids.

Also within the scope of this invention are isolated polynucleotides (e.g., cDNA) encoding the recombinant immunotoxin polypeptides of the invention and their homologs, and in particular, polynucleotides encoding sc(UCHT-1)-PE38 having residues 1-601, 2-601 or 3-601 of SEQ. ID NO:1, or fragments of sc(UCHT-1)-PE38 having at least 100 (and preferably at least 200) amino acids.

This invention includes not only the nucleic acid depicted in SEQ. ID NO:2, but also isolated nucleic acids encoding the polypeptide of SEQ. ID. NO:1 or a fragment thereof and having a sequence which differs from the nucleotide sequence shown in SEQ. ID NO:2 due to the degeneracy of the genetic code; as well as complementary strands of the foregoing nucleic acids.

Another aspect of the invention provides a polynucleotide (having preferably at least 300 bases (nucleotides), and more preferably at least 600 bases, and even more preferably at least 900 bases) which hybridizes to a polynucleotide which encodes a polypeptide of the invention, such as the polypeptide of SEQ. ID. NO:1. Said hybridization reaction may be carried out under under low or high stringency conditions.

Appropriate stringency conditions which promote DNA hybridization (for example, 6.0× sodium chloride/sodium nitrate (SSC) at about 45° C. followed by a wash of 2.0× SSC at 50° C.), are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0× SSC at 50° C. to a high stringency of about 0.2× SSC at 50° C. In addition, the temperature in the wash step can be increased from low stringency conditions at room temperature, about 22° C. to high stringency conditions at about 65° C.

By the term “stringent hybridization conditions” is intended overnight incubation at 42° C. in a solution comprising: 50% formamide, 5× SSC 750 mM NaCl, 75 mM trisodium citrate, 50 mM sodium phosphate (pH 7.6), 5× Denhardt's solution, 10% dextran sulfate, and 20 mu g/ml denatured, sheared salmon sperm DNA, followed by washing the filters in 0.1× SSC at about 65° C.

By “isolated” polynucleotide(s) is intended a nucleic acid molecule, DNA or RNA, which has been removed from its native environment. For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

The invention also includes isolated oligonucleotides encoding the connector peptides and/or linker of the invention. Such oligonucleotides should be “fused in frame” with the polynucleotides encoding the CD3-binding domain and PE component, and preferably include restriction sites unique in the molecule.

By “fused in frame” is meant that: (1) there is no shift in reading frame of the CD3-binding domain or the PE component caused by the linker oligonucleotide; and (2) there is no translation termination between the reading frames of the CD3-binding domain and the PE component.

This invention further encompasses physiologically functional equivalent proteins of the novel fusion polypeptides which are intermediates in the synthesis of the novel polypeptides.

The term “physiologically functional equivalent” refers to a larger molecule comprising the fusion polypeptide of the invention to which has been added such amino acid sequence as is necessary or desirable for effective expression and secretion of the mature recombinant fusion polypeptide of the invention from a particular host cell.

Such added sequence is typically at the amino terminus of the mature protein, and usually constitutes a leader (i.e. signal) sequence which serves to direct the proteins into the secretory pathway, and is normally cleaved from the protein at or prior to secretion of the protein from the cell.

The signal sequence can be derived from the natural N-terminal region of the relevant protein, or it can be obtained from host genes coding for secreted proteins, or it can derive from any sequence known to increase the secretion of the polypeptide of interest, including synthetic sequences and all combinations between a “pre” and a “pro” region. The juncture between the signal sequence and the sequence encoding the mature protein should correspond to a site of cleavage in the host.

In the polypeptides of the invention wherein a CD3-binding region leads expression, i.e. is upstream from other coding sequences in the fusion molecule, it may be expedient to utilize a signal sequence to effectively obtain expression from mammalian systems (e.g., CHO, COS), or yeast (e.g., P. pastoris).

However, the additional signal sequence is not necessarily that of the native immunoglobulin chain and may be obtained from any suitable source, provided it is suitable to effect expression/secretion of the mature polypeptide from the particular host cell.

The addition of other sequences for facilitation of purification at the amino or carboxy terminus of the protein is contemplated as part of the invention. Examples of such sequences include poly-histidine tags for purification on nickel affinity resins and peptide sequences for recognition by antibodies against c-myc, or hemagglutinin (HA). Such peptide “tags” are familiar to those skilled in the art.

In immunotoxin polypeptides of the invention wherein a PE toxin component leads expression, a suitable leader sequence may comprise the native PE exotoxin A leader sequence (SEQ. ID. NO:4) to accomplish secretion of the mature heterologous polypeptide from E.coli, mammalian (e.g., CHO, COS) cells or yeast. However, other leader sequence, not necessarily native to PE or to the host cell, may provide effective expression of the mature fusion protein in certain hosts.

4. Methods for Preparation of Recombinant Immunotoxins of the Invention.—In General.

a. Preparation of Antibody Derived CD3-Binding Moiety.

The general strategy for cloning one or more regions of an antibody begins by extracting RNA from the hybridoma cells, and reverse transcribing the RNA using random hexamers as primers.

In particular, in order to clone the Fv fragment of an antibody, each of the V_{H} and V_{L} domains is amplified by polymerase chain reactions (PCR). Heavy chain sequences can be amplified using 5′-end primers designed according to the amino-terminal protein sequences of the heavy chain and 3′ primers according to consensus immunoglobulin constant region sequences (Kabat and Wu, supra).

Light chain Fv regions are amplified using 5′-end primers designed according to the amino-terminal protein sequences of the antibody light chain, and in combination with the primer C-kappa. Suitable primers for isolating the Fv region of UCHT-1 are illustrated in Table I of Example 1, although one of skill in the art would recognize that other suitable primers may be derived from the sequence listings provided herein.

The crude PCR products are subcloned into a suitable cloning vector. Clones containing the correct size insert by DNA restriction are identified. The nucleotide sequence of the heavy or light chain coding regions may then be determined from double stranded plasmid DNA using sequencing primers adjacent to the cloning site. Commercially available kits (e.g., the Sequenase kit, U.S. Biochemical Corp., Cleveland, Ohio, USA) may be used to facilitate sequencing the DNA.

It will also be appreciated that, given the sequence information disclosed herein, one of ordinary skill in the art may readily prepare nucleic acids encoding these sequences using well-known methods. Thus, DNA encoding the Fv regions may be prepared by any suitable method, including, for example, amplification techniques such as ligase chain reaction (LCR) and self-sustained sequence replication, cloning and restriction of appropriate sequences or direct chemical synthesis, such as by the phosphotriester method, the phosphodiester method, the diethylphosphoramidite method and the solid support method. Chemical synthesis produces a single stranded oligonucleotide. This may be converted into double stranded DNA by hybridization with a complementary sequence, or by polymerization with a DNA polymerase using the single strand as a template. While it is possible to chemically synthesize an entire single chain Fv region, it is preferable to synthesize a number of shorter sequences (about 100 to 150 bases) that are later ligated together.

Alternatively, subsequences may be cloned and the appropriate subsequences cleaved using appropriate restriction enzymes. The fragments may then be ligated to produce the desired DNA sequence.

Once the Fv variable light and heavy chain DNA is obtained, the sequences may be ligated together, either directly or through a DNA sequence encoding a peptide linker, or by PCR, using techniques well known to those of skill in the art. In a preferred embodiment, heavy and light chain regions are connected by a flexible peptide linker which starts at the carboxyl end of the light chain Fv domain and ends at the amino terminus of the heavy chain Fv domain. The entire sequence encodes the Fv domain in the form of a single-chain CD3-binding moiety.

b. Fusion of CD3-Binding Region and PE Component.

The Fv region may be fused directly to the toxin moiety or may be joined through a connector peptide. The connector peptide may be employed simply to provide space between the antibody and the toxin moiety or to facilitate mobility between these regions to enable them to each attain their optimum conformation. The DNA sequence comprising the connector peptide may also provide sequences (such as primer sites or restriction sites) to facilitate cloning or may preserve the reading frame between the sequence encoding the antibody and the toxin moiety.

In general, the cloning of an immunotoxin fusion protein according to the invention involves separately preparing the DNA encoding the CD3-binding moiety and the DNA encoding the PE toxin moiety, and recombining the DNA sequences in a plasmid or other vector to form a construct encoding the particular desired fusion protein. The vector can be an expression plasmid containing appropriate promoter sequence, etc., or the immunotoxin-encoding DNA fragment can be subsequently transferred into an expression plasmid. Another approach involves inserting the DNA encoding the CD3-binding moiety into a construct already encoding the PE toxin moiety.

c. Expression of Recombinant Immunotoxin.

Proteins of the invention can be expressed in a variety of host cells, including E. coli, other bacterial hosts, yeast, and various higher eucaryotic cells such as the COS, CHO and HeLa cell lines and myeloma cell lines. The recombinant protein gene will be operably linked to appropriate expression control sequences for each host. For E. coli, this includes a promoter such as the T7, trp, tac, lac or lambda promoters, a ribosome binding site, and preferably a transcription termination signal. For eucaryotic cells, the control sequences will include a promoter and preferably an enhancer derived form immunoglobulin genes, SV40, cytomegalovirus, etc., and a polyadenylation sequence, and may include splice donor and acceptor sequences.

Both diphtheria toxin and Pseudomonas exotoxin prevent protein synthesis in eucaryotic cells by ADP-ribosylation of elongation factor-2 (EF-2), an essential eucaryotic translation factor. Therefore, for eucaryotic expression, it is preferable that cells in which EF-2 is mutated and therefore resistant to ADP-ribosylation by P. exotoxin be utilized. Such mutant hosts and mutant EF-2 proteins have been described for both mammalian (Moehring et al., 1979 Somatic Cell Genetics 5: 469-480; Kohno et al., 1987, J. Biol. Chem. 262: 12298-12305) and yeast cells (Phan et al., 1993, J. Biol. Chem. 268:8665-8668; Kimata, et al., 1993, Biochem. Biophys. Res. Commun. 191: 1145-1151).

The plasmids of the invention can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

It is apparent that modifications can be made to the single chain Fv region and fusion proteins comprising the single chain Fv region without diminishing their biological activity. Some modifications may be made to facilitate the cloning, expression, or incorporation of the single chain Fv region into a fusion protein. Such modifications are well known to those of skill in the art and include, for example, a methionine added at the amino terminus to provide an initiation site, or additional amino acids placed on either terminus to create conveniently located restriction sites or termination codons. For example, the primers used in Example 1 introduce a sequence encoding an initiator methionine for expression in E. coli, and BamHI, XbaI, SalI, NcoI and BstXI restriction sites to facilitate cloning.

Once expressed, the recombinant proteins can be purified according to standard procedures of the art, including ammonium sulfate precipitation, affinity columns, column chromatography, gel electrophoresis, and the like.

Substantially pure compositions of at least about 90 to 95% homogeneity are preferred, and compositions having 98 to 99%, or greater than 99%, homogeneity are most preferred for pharmaceutical uses. Once purified, partially or to homogeneity as desired, the polypeptides should be substantially free of endotoxin for pharmaceutical purposes and may be used therapeutically.

One of skill in the art would recognize that after chemical synthesis, biological expression, or purification, the single chain Fv region or a fusion protein comprising a single chain Fv region may possess a conformation substantially different from that of the native protein. In this case, it may be necessary to denature and reduce the protein and then to cause the protein to re-fold into the preferred conformation.

Methods for expressing single chain antibodies and/or denaturing the protein and inducing refolding to an appropriate folded form, including single chain antibodies, from bacteria such as E. coli, have been described and are well-known and are applicable to the polypeptides of this invention. See, Buchner et al., Analytical Biochemistry 205:263-270(1992).

In particular, functional protein from E. coli or other bacteria is often generated from inclusion bodies and requires the solubilization of the protein using strong denaturants, and subsequent refolding. In the solubilization step, a reducing agent must be present to dissolve disulfide bonds as is well-known in the art. An exemplary buffer with a reducing agent is: 0.1 M Tris, pH 8, 6M guanidine, 2mM EDTA, 0.3M DTE (dithioerythritol). Reoxidation of protein disulfide bonds can be effectively catalyzed in the presence of low molecular weight thiol reagents in reduced and oxidized form, as described by Buchner et al.(1992), supra.

Renaturation is typically accomplished by dilution (e.g., 100-fold) of the denatured and reduced protein into refolding buffer. Renaturation in the presence of 8 mM GSSG has been found to provide a reproducible, highly stable product. An exemplary buffer for this purpose is 0.1 M Tris, pH 8.0, 0.5 M L-arginine, 8 mM oxidized glutathione (GSSG), and 2 mM EDTA.

5. Therapeutic Uses of Recombinant Anti-CD3 Immunotoxins.

The immunotoxin polypeptides described herein are utilized to effect at least partial T-cell depletion in order to treat or prevent T-cell mediated diseases or conditions of the immune system. The immunotoxins may be utilized in methods carried out in vivo, in order to systemically reduce populations of T cells in a patient. The immunotoxins may also be utilized ex vivo in order to effect T-cell depletion from a treated cell population.

In Vivo Applications

It is within the scope of the present invention to provide a prophylaxis or treatment of T-cell mediated diseases or conditions by administering immunotoxin to a patient in vivo for the purpose of systemically killing T cells in the patient, and as a component of a preparation or conditioning regimen or induction tolerance treatment in connection with bone marrow or stem cell transplantation, or solid organ transplantation from either a human (allo-) or non-human (xeno-) source.

Both B and T lymphocytes originate in the bone marrow from a common lymphoid progenitor, the pluripotent stem cell, but only B lymphocytes mature in the bone marrow. The T lymphocytes migrate to the thymus to undergo maturation, and then enter the bloodstream, from which they migrate to the peripheral lymphoid tissues. The lymphoid tissues include the central lymphoid organs where lymphocytes are generated, and secondary or peripheral lymphoid organs, where adaptive immune responses are initiated. The central lymphoid organs are the bone marrow and thymus. The peripheral lymphoid organs include the lymph nodes, the spleen, the gut-associated lymphoid tissues, the bronchial-associated lymphoid tissue and mucosal-associated lymphoid tissue. Janeway and Travers, supra, at §1-2.

This invention comprises a method of treatment or prophylaxis of T-cell mediated disorders in a patient, comprising administering to a patient in need thereof a T-cell depleting effective amount of an immunotoxin of the invention.

Depletion of the levels of T cells in the bone marrow, the peripheral blood and/or lymphoid tissues of the patient can ameliorate the patient's T-cell mediated response to antigen, and assist in tolerance induction.

For example, the immunotoxins can usefully be administered to a patient who is or will be a recipient of an allotransplant (or xenotransplant), in order to effect T-cell depletion in the patient and thereby prevent or reduce T-cell mediated acute or chronic transplant rejection of the transplanted allogeneic (or xenogeneic) cells, tissue or organ in the patient, or to permit the development of immunological tolerance to the cells, tissue or organ.

Preferably, when administered in vivo to prevent or treat organ transplant rejection, it is desirable that the immunotoxin be administered to the patient over time in several doses. In general, it is preferred that at least the first dose precede the transplant surgery (preferably as long in advance as possible), and a subsequent dose or doses begin at the time of or shortly following the surgery.

The immunotoxins can be administered in vivo either alone or in combination with other pharmaceutical agents effective in treating acute or chronic transplant rejection including cyclosporin A, cyclosporin G, rapamycin, 40-O-2-hydroxyethyl-substituted rapamycin (RAD), FK-506, mycophenolic acid, mycophenolate mofetil (MMF), cyclophosphamide, azathioprene, brequinar, leflunamide, mizoribine, deoxyspergualines, 2-amino-2-[2-(4-octylphenyl)ethyl]propane-1,3-diol hydrochloride (FTY 720), corticosteroids (e.g., methotrexate, prednisolone, methylprednisolone, dexamethasone), or other immunomodulatory compounds (e.g., CTLA4-Ig); anti-LFA-1 or anti-ICAM antibodies, or other antibodies that prevent co-stimulation of T cells, for example antibodies to leukocyte receptors or their ligands (e.g., antibodies to MHC, CD2, CD3, CD4, CD7, CD25, CD28, B7, CD40, CD45, CD58, CD152 (CTLA-4), or CD 154 (CD40 ligand).

In particular, prolonged graft acceptance and even apparent immunologic tolerance can be achieved by combined administration of an anti-CD3 immunotoxin of the invention and a spergualin derivative, such as a deoxyspergualine compound, or other spergualin analog, and this invention in a preferred embodiment comprises the combined administration of anti-CD3 immunotoxin and a deoxyspergualine compound in a tolerance induction regimen, see for example, Eckhoff et al., abstract presented to American Society of Transplant Surgeons, May 15, 1997, and Contreras, et al., (1998) Peritransplant tolerance induction with anti-CD3 immunotoxin : A matter of proinflammatory cytokine control. Transplantation 65: 1159, both incorporated by reference. The term “deoxyspergualine compound” includes 15-deoxyspergualin (referred to as “DSG”, and also known as gusperimus), i.e. i.e. N-[4-(3-aminopropyl) aminobutyl]-2-(7-N-guanidinoheptanamido)-2-hydroxyethanamide, and its pharmaceutically acceptable salts, as disclosed in U.S. Pat. No. 4,518,532, incorporated by reference; and in particular (−)-15-deoxyspergualin and its pharmaceutically acceptable salts as disclosed in U.S. Pat. No. 4,525,299, incorporated by reference. The optically active (S)-(−) or (R)-(+)-15-deoxyspergualin isomers and salts thereof are disclosed in U.S. Pat. No. 5,869,734 and EP 765,866, both incorporated by reference; and the trihydrochloride form of DSG is disclosed in U.S. Pat. No. 5,162,581, incorporated by reference.

Other spergualin derivatives for use with anti-CD3 immunotoxin in a tolerance induction regimen include compounds disclosed in U.S. Pat. Nos. 4,658,058, 4,956,504, 4,983,328, 4,529,549,; and EP 213,526, EP 212,606, all incorporated by reference.

The invention in a further preferred embodiment comprises the combined administration of an anti-CD3 immunotoxin according to the invention and still other spergualin analogs, such as compounds disclosed in U.S. Pat. No. 5,476,870 and EP 600,762, both incorporated by reference, e.g.,

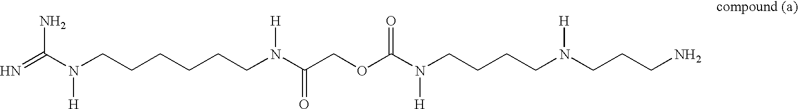

i.e. 2-[[[4-[[3-(Amino)propyl]amino]butyl]amino]carbonyloxy]-N-[6-[(aminoiminomethyl)amino]hexyl]acetamide (“tresperimus”) and

its pharmaceutically acceptable addition salts with a mineral or organic acid;

compounds disclosed in U.S. Pat. No. 5,637,613 and EP 669,316, both incorporated by reference, e.g.,

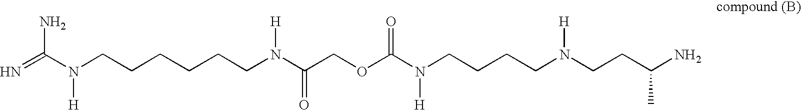

i.e. 2-[[[4-[[3(R)-(Amino)butyl]amino]butyl]amino carbonyloxy]-N-[6-[(aminoiminomethyl)amino]hexyl]acetamide tris (trifluoroacetate) and other pharmaceutically acceptable salts thereof. Pharmaceutically acceptable salts of the above compounds include salts with a mineral acid or an organic acid, including (with respect to mineral acids) hydrochloric, hydrobromic, sulfuric and phosphoric acid, and (with respect to organic acids) fumaric, maleic, methanesulfonic, oxalic and citric;

compounds disclosed in U.S. Pat. No. 5,733,928 and EP 743,300, both incorporated by reference;

compounds disclosed in U.S. Pat. No. 5,883,132 and EP 755,380, both incorporated by reference; and

compounds disclosed in U.S. Pat. No. 5,505,715 (e.g., col. 4, 1. 44-col. 5 , 1. 45), incorporated by reference.

By “combined administration” is meant treatment of the organ transplant recipient with both an anti-CD3 immunotoxin of the invention and the spergualin derivative or analog.

Administration of the immunotoxin and the spergualin derivative or analog need not be carried out simultaneously, but rather may be separated in time. Typically, however, the course of administration of the immunotoxin and the spergualin related compound will be overlapping to at least some extent.

The total dose of the anti-CD3 immunotoxin is preferably given over 2-3 injections, the first dose preceding the transplant by the maximal time practicable, with subsequent injections spaced by intervals of, for example, about 24 hours.

The immunotoxin is preferably administered prior to transplant and at the time of and/or following transplant.

In allotransplantation, administration of the anti-CD3 immunotoxin preferably precedes transplant surgery by about 2-6 hours, whereas for xenotransplantation or living related allotransplantation, the first anti-CD3 immunotoxin injection may precede transplantation by as much as one week, see for example, Knechtle, S. J., et al. (1997) FN18-CRM9 immunotoxin promotes tolerance in primate renal allografts. Transplantation 63: 1.

In a tolerance induction regimen, the immunotoxin treatment is preferably curtailed no later than about 14 days, and preferably on about day 7 , or on day 5, or even on day 3, post-transplant.

The spergualin derivative or analog may be administered prior to transplant, at the time of transplant, and/or following transplant. The length of treatment either before or after transplant may vary.

In a tolerance induction regimen, the treatment with spergualin derivative or analog compound is preferably withdrawn not later than about 120 days following transplant, and more preferably after about 60 days post-transplant, and more preferably after about 30 days, and even more preferably not later than 14, or even about 10 days, post-transplant.

Thus, the term “combined administration” includes within its scope a treatment regimen wherein, for example, one or more doses of immunotoxin is/are administered prior to the transplant, followed by one or more doses commencing at around the time of transplant; together with administration of the spergualin derivative or analog also prior to and/or at the time of transplant, and typically continuing after transplant.

Corticosteroids such as methylprednisolone may be incorporated into the combined administration regimen. For example, steroid administration may commence prior to transplant, and may continue with one or more doses thereafter.

The anti-CD3 immunotoxin of the invention is preferably provided in a dose sufficient to reduce the T-cell number in a patient by 2-3 logs.

A total effective dosage to reduce the T-cell number in a patient by 2-3 logs in accordance herewith may be between about 50 μg/kg and about 10 mg/kg body weight of the subject, and more preferably between about 0.1 mg/kg and 1 mg/kg.

A dosage regimen for an induction treatment with the spergualin derivative or analog may be between 1 and 10 mg/kg/day for 0-30 days, optimally, for example about 2.5 mg/kg/day for 15 days.

Additional steroids may be administered at the time of the anti-CD3 immunotoxin injections, for example as a decreasing regimen of methylprednisone, such as 7 mg/kg on the day of the transplant surgery, 3.5 mg/kg at +24 hours, and 0.35 mg/kg at +48 hours. Alternatively, the steroid dosage may be held constant, for example treatment with 40 mg/kg of prednisone at the time of immunotoxin injection. It is understood that the exact amount and choice of steroid can vary, consistent with standard clinical practice.

In a preferred embodiment of the combination therapy of the invention, the immunotoxin of the combined therapy is scFv (UCHT-1)-PE38, and is in particular an immunotoxin having SEQ. ID. No:1.

Said scFv(UCHT-1)-PE38 is preferably co-administered with 15-deoxyspergualine, and especially, (−)-15-deoxyspergualine.

In another aspect, said scFv(UCHT-1)-PE38 is co-administered with the abovementioned compound (a).

In a still further embodiment, said scFv(UCHT-1)-PE38 is co-administered with the abovementioned compound (b).

In the practice of the above combination therapy and the other methods of this invention in the context of xenotransplantation, and especially where the transplant recipient is human, the donor cells, tissues or organs are preferably porcine, and are most preferably recruited from transgenic, e.g., human DAF expressing, pigs.

In another embodiment of the methods of the invention, the immunotoxins can be administered in vivo to a bone marrow recipient for prophylaxis or treatment of host-versus-graft disease through killing of host (i.e. bone marrow transplant recipient) T cells. Marrow transplants become necessary in the treatment of certain diseases, such as leukemia, aplastic anemia or certain genetic disorders, in which the patient's own marrow is severely flawed or where total body irradiation or chemotherapy have destroyed the patient's hematopoietic system. Absent reconstitution of the hematopoietic system by bone marrow transplantation, the patient becomes severely immunodepressed and susceptible to infection.

Stable engraftment of donor allogeneic bone marrow depends in large part on MHC matching between donor and recipient. In general, mismatching only to the extent of one or two antigens is tolerable in bone marrow transplantation because of rejection of the disparate bone marrow graft by recipient T cells. (Also, graft versus host disease, discussed below, is very severe when there are greater disparities.) In addition, even minor mismatching conventionally necessitates conditioning of the recipient by lethal or sublethal doses of total body irradiation or total lymphoid irradiation to deplete recipient T-cells. This requirement for irradiation of the bone marrow transplant patient which renders the patient totally or nearly immunoincompetent poses a significant limitation on clinical application of bone marrow transplantation to a variety of disease conditions in which it is potentially useful, including solid organ or cellular transplantation, sickle cell anemia, thalassemia and aplastic anemia.

The present invention addresses this problem by providing a directed means of killing recipient T cells in the absence of radiation.

Thus this invention provides in another of its aspects, a method for conditioning a bone marrow transplant patient prior to engraftment in the patient of donor bone marrow and/or stem-cell enriched peripheral blood cells, comprising administration of a T-cell depleting effective amount of immunotoxin to the patient. The immunotoxin effects reductions in the T cell population in the patient and thereby exerts a prophylaxis against host (i.e. the patient's) rejection of the donor bone marrow graft. Methods of obtaining donor compositions enriched for hematopoietic stem cells are disclosed in U.S. Pat. No. 5,814,440, No. 5,681,559, No. 5,677,136, and No. 5,061,620, all incorporated by reference.

Graft-versus-host disease (GVHD), in particular, is a sometimes fatal, often debilitating complication of allogeneic bone marrow transplant which is mediated primarily, if not exclusively, by T lymphocytes. GVHD is caused by donor T cells which are acquired in the graft by the bone marrow recipient and which develop an immune response against the host. GVHD typically results from incomplete immunologic matching of donor and recipient Human leukocyte antigens (HLA).

Accordingly, this invention also contemplates a method of prophylaxis or treatment of GVHD in a bone marrow transplant patient, comprising administration of an immunotoxin of the invention to the patient during the early post-transplant period, or when symptoms of GVHD become manifest, in an amount sufficient to effect reductions in levels of T cells in the host (i.e. patient), including both donor and host T cells. The early depletion of donor and host T-cells also facilitates the development of allogeneic chimerism; that is, the T cells which are given space to mature following host T-cell ablation by immunotoxin are rendered tolerant of both donor and host antigens and do not participate in graft versus host rejection. By “early post-transplant period” is meant a period of one or more days up to about two weeks following bone marrow transplantation.

In a further embodiment, the anti-CD3 immunotoxin of the invention can be administered to a patient in need thereof to treat still other T-cell mediated pathologies, such as T-cell leukemias and lymphomas. As mentioned above, clinical treatment of T-cell leukemias and lymphomas typically relies on whole body irradiation to indiscriminately kill lymphoid cells of a patient, followed by bone marrow replacement. An immunotoxin of the invention administered to a patient suffering from leukemia/lymphoma can replace whole body radiation with a selective means of eliminating T-cells.

In additional aspects of the invention, the immunotoxins of the invention may also be administered to a patient in vivo to treat T-cell-mediated autoimmune disease, such as systemic lupus erythematosus (SLE), type I diabetes, rheumatoid arthritis (RA), myasthenia gravis, and multiple sclerosis, by ablating populations of T cells in the patient.

The immunotoxins can also be administered to a subject afflicted with an infectious disease of the immune system, such as acquired immune deficiency syndrome (AIDS), in an amount sufficient to deplete the patient of infected T-cells and thereby inhibit replication of HIV-1 in the patient.

Additionally, the anti-CD3 immunotoxin can be administered to patients to treat conditions or diseases in instances in which chronic immunosuppression is not acceptable, e.g., by facilitating islet or hepatocyte transplants in patients with diabetes or metabolic diseases, respectively. Diseases and susceptibilities correctable with hepatocyte transplants include hemophilia, α1-antitrypsin insufficiencies, and hyperbilirubinemias.

In the above methods of the invention, the patient is preferably human and the donor may be allogeneic (i.e. human) or xenogeneic (e.g., swine). The transplant may be an unmodified or modified organ, tissue or cell transplant, e.g., heart, lung, combined heart-lung, trachea, liver, kidney, pancreas, Islet cell, bowel (e.g., small bowel), skin, muscles or limb, bone marrow, oesophagus, cornea or nervous tissue transplant.

For in vivo applications, the immunotoxin will be administered to the patient in an amount effective to kill at least a portion of the targeted population of CD3-bearing cells (i.e. T-cells).

In general, an effective amount of immunotoxin will deplete a targeted population of T cells, i.e. in the lymph system and/or peripheral blood, by 1 or more logs, and more preferably by at least about 2 logs, and even more preferably by at least 2-3 logs. The most effective mode of administration and dosage regimen depends on the severity and course of the disease, the subject's health and response to treatment and the judgment of the treating physician. Thus the dosages of the molecules should be titrated to the individual subject.

Preferably, in the treatment or prophylaxis of GVHD accompanying bone marrow transplantation, the immunotoxin is administered to the bone marrow transplant recipient in an amount sufficient to reduce the total T-cell population (i.e. donor plus recipient T cells) present in the patient blood and lymph nodes immediately following bone marrow transplantation by at least about 50% and more preferably at least about 80%, and even more preferably at least about 95% (e.g., 99%), i.e. by at least 2 logs (e.g., by 2-3 logs).

A suitable dosing regimen for a bone marrow recipient, to treat or prevent host versus graft disease and/or GVHD, may comprise administration of immunotoxin immediately prior to, and/or immediately following bone marrow transplantation on each alternating day over the course of six days after transplant, to bring the total dose to about 10-500 μg/kg, and more preferably 200-300 μg/kg.

For treatment of leukemia/lymphoma, the immunotoxin is administered in an amount sufficient to reduce the T-cell population at the time of administration by at least about 50%, and more preferably at least about 80%, and more preferably at least about 95% (e.g., 99%), i.e. by at least 2 logs (e.g., by at least 2-3 logs).

The levels of CD3-bearing cells, and in particular, of T cells, in the patient's bone marrow, blood or lymphoid tissues, can be assayed by FACS analysis.

The effectiveness of immunotoxin treatment in depleting T-cells from the peripheral blood and lymphoid organs can be determined by comparing T-cell counts in blood samples and from macerated lymphoid tissue taken from the subject before and after immunotoxin treatment. Depletion of T-cells can be followed by flow cytometry as described by Neville et al., 1996, J. Immunother. 19:95-92.

Depletion of T-cell numbers by 2 logs, by a chemically conjugated immunotoxin comprised of an anti-rhesus CD3 monoclonal antibody conjugated to a cell binding domain-deleted form of diphtheria toxin, has been shown to be associated with transplantation tolerance to renal allografts in rhesus monkeys (Thomas et al., 1997, Transplantation 64:124-135; Knechtle et al., 1997, Transplantation 63:1-6).

In general, a total effective dosage to reduce the T-cell number in a patient by 2-3 logs in accordance herewith can best be described as between about 50 μg/kg and about 10 mg/kg (e.g., between about 50 μg/kg and 5 mg/kg) body weight of the subject, and more preferably between about 0.1 mg/kg and 1 mg/kg.

The patient may be treated on a daily basis in single or multiple administrations.

The immunotoxin composition may also be administered on a per month basis (or at such weekly intervals as may be appropriate), also in either single or multiple administrations.

It is envisaged that, in the course of the disease state, the dosage and timing of administration may vary. Initial administrations of the composition may be at higher dosages within the above ranges, and administered more frequently than administrations later in the treatment of the disease.

For example, the polypeptide, scFv(UCHT-1)-PE38 of Example 1, may be administered to a kidney transplant patient starting just prior to transplantation and continuing, post-transplant, over the course of a week in daily or alternate day dosing, at a dose of about 0.3-10 mg per week of polypeptide in the average patient (70 kg). After the first week post-transplant, the treatment regimen may be reduced to alternating weeks, with dosages ranging from 0.1 mg to 1 mg of polypeptide per week in the average patient. It is expected, however, that immunotoxin treatment shall be curtailed at five weeks after transplant, and more typically at three weeks, or even at one week post-transplant.

Ex Vivo Applications

It is also within the scope of the present invention to utilize the immunotoxins for purposes of ex vivo depletion of T cells from isolated cell populations removed from the body.

In one aspect, the immunotoxins can be used in a method for prophylaxis of organ transplant rejection, wherein the method comprises perfusing the donor organ (e.g., heart, lung, kidney, liver) prior to transplant into the recipient with a composition comprising a T-cell depleting effective amount of immunotoxin, in order to purge the organ of sequestered donor T-cells.

In another embodiment of the invention, the immunotoxins can be utilized ex vivo in an autologous therapy to treat T cell leukemia/lymphoma or other T-cell mediated diseases or conditions by purging patient cell populations (e.g., bone marrow) of cancerous or otherwise affected T-cells with immunotoxin, and reinfusing the T-cell-depleted cell population into the patient.

In particular, such a method of treatment comprises:

(a) recruiting from the patient a cell population comprising CD3-bearing cells (e.g., bone marrow);

(b) treating the cell population with a T-cell depleting effective amount of immunotoxin; and

(c) infusing the treated cell population into the patient (e.g., into the blood).

A still further application of such an autologous therapy comprises a method of treating a subject infected with HIV, comprising the steps of:

(a) isolating a cell population from the patient comprising T cells infected with HIV.

(b) treating the isolated cell population with a T-cell-depleting effective amount of immunotoxin; and

(c) reintroducing the treated cell population into the patient.

According to still another embodiment of the invention, the immunotoxins can be utilized ex vivo for purposes of effecting T cell depletion from a donor cell population as a prophylaxis against graft versus host disease, and induction of tolerance, in a patient to undergo a bone marrow transplant. Such a method comprises the steps of:

(a) providing a cell composition comprising isolated bone marrow and/or stem cell-enriched peripheral blood cells of a suitable donor (i.e. an allogeneic donor having appropriate MHC, HLA-matching);

(b) treating the cell composition with an effective amount of immunotoxin to form an inoculum at least partially depleted of viable CD3-bearing cells (i.e. T-cells); and

(c) introducing the treated inoculum into the patient.

By virtue of T-cell depletion from the donor inoculum, the donor T cells which mature following engraftment are rendered immunologically tolerant of the host and will not initiate graft versus host rejection.

Advantageously, for purposes of the above-described ex vivo therapies, the immunotoxin can be provided in a therapeutic concentration far in excess of levels which could be accomplished or tolerated in vivo.

For example, the immunotoxin may be incubated with CD3-expressing cells in culture at a concentration of about 0.5 to 50,000 ng/ml in order to kill CD3-bearing cells in said culture.

Thus, it has been found that incubation of human cytokine-mobilized peripheral blood leukocytes (CMPBL, 5×10⁶/ml) in culture medium for one hour at 25° C. with 0.005 to 50 μg/ml of the immunotoxin prepared in Example 1, results in depletion of the number of CD3⁺ cells by about 2.5 logs, and reduces PHA-induced proliferation to background levels as measured by ³H-thymidine uptake.

In a further aspect, the above ex vivo therapeutic methods can be combined with in vivo administration of immunotoxin, to provide improved methods of treating or preventing rejection in bone marrow transplant patients, and for achieving immunological tolerance.

For example, a method comprising both in vivo and ex vivo administration of an immunotoxin of the invention for the prophylaxis and/or treatment of host versus graft disease and/or graft versus host disease in a patient to undergo a bone marrow transplant comprises the steps of:

(a) reducing the levels of viable CD3-bearing cells (i.e. T cells) in the patient (i.e. from the patient's peripheral blood or lymph system);

(b) providing an inoculum comprising hematopoietic cells (i.e. bone marrow and/or stem cell-enriched peripheral blood cells) of a suitable donor treated with a T-cell depleting effective amount of immunotoxin; and

(c) introducing the inoculum into the patient, and thereafter optionally administering immunotoxin to the patient to further deplete donor and patient T cells.

Step (a), i.e. depletion of patient T cells can be carried out by in vivo administration of immunotoxin to the patient and/or by an autologous therapy comprising ex vivo treatment of isolated patient bone marrow or peripheral blood with immunotoxin, as previously described.

The in vivo and ex vivo methods of the invention as described above are suitable for the treatment of diseases curable or treatable by bone marrow transplantation, including leukemias, such as acute lymphoblastic leukemia (ALL), acute nonlymphoblastic leukemia (ANLL), acute myelocytic leukemia (AML), and chronic myelocytic leukemia (CML), cutaneous T-cell lymphoma, severe combined immunodeficiency syndromes (SCID), osteoporosis, aplastic anemia, Gaucher's disease, thalassemia, mycosis fungoides (MF), Sezany syndrome (SS), and other congenital or genetically-determined hematopoietic abnormalities.

In particular, it is also within the scope of this invention to utilize the immunotoxins as agents to induce donor-specific and antigen-specific tolerance in connection with allogeneic or xenogeneic cell therapy or tissue or organ transplantation. Thus, the immunotoxin can be administered as part of a conditioning regimen to induce immunological tolerance in the patient to the donor cells, tissue or organ, e.g., heart, lung, combined heart-lung, trachea, liver, kidney, pancreas, Islet cell, bowel (e.g., small bowel), skin, muscles or limb, bone marrow, oesophagus, cornea or nervous tissue.

Systemic donor-specific transplantation tolerance has been transiently achieved in MHC-mismatched animal models as well as in humans through chimerism as a result of total lymphoid irradiation of a recipient followed by bone marrow transplantation with donor cells. The reconstituted animals exhibit stable mixed multilineage chimerism in their peripheral blood, containing both donor and recipient cells of all lymphohematopoietic lineages, including T cells, B cells, natural killer cells, macrophages, erythrocytes and platelets. Furthermore, the mixed allogeneic chimeras display donor-specific tolerance to donor-type skin grafts, while they readily reject third-party grafts. Donor-specific tolerance is also confirmed by in vitro assays in which lymphocytes obtained from the chimeras are shown to have diminished proliferative and cytotoxic activities against allogeneic donor cells, but retain normal immune reactivity against third-party cells.

Thus the present invention further contemplates a method of conditioning a patient to be transplanted with donor cells, or a tissue or organ. The method comprises the steps of:

(a) reducing levels of viable CD3-bearing (i.e. T cells) in the patient (i.e. in the peripheral blood or lymph system of the patient);

(b) providing an inoculum comprising isolated hematopoietic cells (i.e. bone marrow and/or stem-cell enriched peripheral blood cells) of the donor treated with a T-cell depleting effective amount of immunotoxin;

(c) introducing the inoculum into the patient; and thereafter,

(d) transplanting the donor cells, tissue or organ into the patient.

The above method is preferably carried out in the absence of total body irradiation or total lymphoid irradiation, and most preferably, in the absence of any radiation.

6. Compositions Comprising Immunotoxin

The recombinant immunotoxin polypeptide of the invention can be administered as an unmodified polypeptide or its pharmaceutically acceptable salt, in a pharmaceutically acceptable carrier.

As used herein the term “pharmaceutically acceptable salt” refers to salts prepared from pharmaceutically acceptable non-toxic acids to form acid addition salts of an amino group of the polypeptide chain, or from pharmaceutically acceptable non-toxic bases to form basic salts of a carboxyl group of the polypeptide chain. Such salts may be formed as internal salts and/or as salts of the amino or carboxylic acid terminus of the polypeptide of the invention.

Suitable pharmaceutically acceptable acid addition salts are those of pharmaceutically acceptable, non-toxic organic acids, polymeric acids, or inorganic acids.

Examples of suitable organic acids comprise acetic, ascorbic, benzoic, benzensulfonic, citric, ethanesulfonic, fumaric, gluconic, glutamic, hydrobromic, hydrochloric, isethionic, lactic, maleic, malic, mandelic, methanesulfonic, mucic, nitric, oxalic, pamoic, pantothenic, phosphoric, salicylic, succinic, sulfuric, tartaric, p-toluenesulfonic, etc., as well as polymeric acids such as tannic acid or carboxymethyl cellulose. Suitable inorganic acids include mineral acids such as hydrochloric, hydrobromic, sulfuric, phosphoric, nitric acid, and the like.

Examples of suitable inorganic bases for forming salts of a carboxyl group include the alkali metal salts such as sodium, potassium and lithium salts; the alkaline earth salts such as for example calcium, barium and magnesium salts; and ammonium, copper, ferrous, ferric, zinc, manganous, aluminum, manganic salts, and the like. Preferred are the ammonium, calcium, magnesium, potassium, and sodium salts.

Examples of pharmaceutically acceptable organic bases suitable for forming salts of a carboxyl group include organic amines, such as, for example, trimethylamine, triethylamine, tri(n-propyl)amine, dicyclohexylamine, beta(dimethylamino)-ethanol, tris(hydroxymethyl)aminomethane, triethanolamine, beta-(diethylamino)ethanol, arginine, lysine, histidien, N-ethylpiperidine, hydrabamine, choline, betaine, ethylenediamine, glucosamine, methylglucamine, theobromine, purines, piperazines, piperidines, caffeine, procaine, and the like.

Acid addition salts of the polypeptides may be prepared in the usual manner by contacting the polypeptide with one or more equivalents of the desired inorganic or organic acid, such as, for example, hydrochloric acid.

Salts of carboxyl groups of the peptide may be conventionally prepared by contacting the peptide with one or more equivalents of a desired base such as, for example, a metallic hydroxide base e.g., sodium hydroxide; a metal carbonate or bicarbonate base such as, for example, sodium carbonate or sodium bicarbonate; or an amine base such as for example triethylamine, triethanolamine, and the like.

For either in vivo or ex vivo applications, the pharmaceutical compositions of the invention comprise a carrier which is preferably a sterile, pyrogen-free, parenterally acceptable liquid.

Water, physiological saline, aqueous dextrose, and glycols are preferred liquid carriers, particularly (when isotonic) for injectable solutions, or for ex vivo uses.

Compositions comprising the immunotoxin or its salt can be administered systemically, i.e. parenterally (e.g., intramuscularly, intravenously, subcutaneously or intradermally), or by intraperitoneal administration.

Compositions particularly useful for parenteral administration, such as intravenous administration or administration into a body cavity or lumen of an organ will commonly comprise a solution of the fusion protein dissolved in a pharmaceutically acceptable carrier, preferably an aqueous carrier such as buffered saline or the like. These compositions are sterile and generally free of undesirable matter. These compositions may be sterilized by conventional, well-known sterilization techniques. The compositions may also contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of immunotoxin protein in these formulations can vary widely, and will be selected primarily based on fluid volumes, viscosities, body weight and the like in accordance with the particular mode of administration selected and the patient's needs. Actual methods for preparing parenterally administrable compositions will be known or apparent to those skilled in the art and are described in more detail in such publications as Remington's Pharmaceutical Science, 15^{th} ed., Mack Publishing Company, Easton, Pa. (1980).

Pharmaceutical compositions comprising the immunotoxins or their salts can also be used for oral, topical, or local administration, such as by aerosol or transdermally.

Unit dosage forms suitable for oral administration include powder, tablets, pills, capsules and lozenges. It is recognized that the polypeptides, when administered orally, must be protected from digestion, such as by complexing the protein with a composition to render it resistant to acidic and enzymatic hydrolysis or by packaging the protein in an appropriately resistant carrier such as a liposome. Various means of protecting proteins from digestion are known in the art.

Examples of the topical dosage form include sprays, opthalmic solutions, nasal solutions and ointments.

For example, a spray can be manufactured by dissolving the peptide in an appropriate solvent and putting it in a spray to serve as an aerosol for commonly employed inhalation therapy. An opthalmic or nasal solution can be manufactured by dissolving the active ingredient peptide in distilled water, adding any auxiliary agent required, such as a buffer, isotonizing agent, thickener, preservative, stabilizer, surfactant, antiseptic, etc., and adjusting the mixture to pH 4 to 9.

Ointments can also be prepared, e.g., by preparing a composition from a polymer solution, such as 2% aqueous carboxyvinyl polymer, and a base, such as 2% sodium hydroxide, mixing to obtain a gel, and mixing with the gel an amount of purified fusion polypeptide.

The composition may be a lyophilizate prepared by methods well known in the art.

In the practice of the in vivo methods of the present invention, a therapeutically effective amount of a recombinant immunotoxin polypeptide, a pharmaceutically acceptable salt thereof, or a pharmaceutical composition containing same, as described above, is administered to a patient in need thereof.

The following exemplification is presented to illustrate the present invention and provide assistance to one of ordinary skill in making and using the same, and is not intended to be limitative of the scope of the invention.

### EXAMPLE 1

### Preparation of scFv(UCHT-1)-PE38.

(a) Cloning of UCHT-1 Antibody Variable Regions from Hybridoma Cells.

The genes encoding the Fv region of murine anti-human CD3 are amplified by RT-PCR from UCHT-1 hybridoma RNA (Beverley and Callard, 1981) using oligonucleotide primers based upon the published sequence of UCHT-1 scFv (Shalaby et al. (1992), supra, and upon consensus primers described for cloning antibody variable regions (Orlandi et al. (1989) PNAS 86: 3833-3387), as listed in Table I.

Oligos IM34A and IM34B are used to amplify the V_{L} region, and IM-61 and IM-34C are used to amplify the V_{H} fragment. The two amplified fragments are then subcloned into E. coli plasmid vectors (TA Vector, Invitrogen) and their DNA sequences determined.

After determining the cloned DNA sequences, the two molecules are combined into a single pUC18-based plasmid by cutting pUC18 and the subcloned PCR-fragments at the appropriate restriction sites and ligating them together with T4 DNA ligase. This plasmid, containing V_{L} followed by a polylinker which is in turn followed by V_{H}, is cut with XbaI plus SalI. A linker comprised of the two annealed oligos, IM-24A and IM24B, designed to contain complementary ends for these two sites, is inserted between the XbaI and SalI sites. The resultant clone, ‘CloneB’, encodes a single chain immunotoxoin with a linker different than that described in SEQ. ID NO:1. The replacement of this linker with the (Gly₃Ser)₄ (SEQ. ID. NO:5) linker used in scFv(UCHT-1)PE38 is described below. However, it was first necessary to investigate two changes in the variable region sequences which are observed relative to the sequence of the clone Fv fragment reported in Shalaby, et al., supra:

(1) a change of A to C at nucleotide position 208 in the heavy chain sequence (V_{H}). This is likely to reflect an error by Shalaby et al. (1992), supra, since the amino acid (Leu) reportedly encoded at this position, does not correlate with the nucleotide sequence in the paper but does correlate with the sequence of the presently obtained clone; and

(2) a change of Phe to Ser at amino acid residue 98. This appears to be a PCR-induced error, and this point mutation in V_{L} is corrected using a standard 4-way PCR reaction in which the desired nucleotide change is incorporated using complementary oligos VL2 and VL3. Flanking oligos, VL1 on the 5′ side and VH4 on the 3′ side, stabilize the change, as described below.

a1. Correction of Point Mutation in V_{L}

PCR reactions using pUC18/UCHT-1 ‘Clone B’ as template are set up with oligo pairs VL1 and VL2 or VL3 and VH4. The two distinct PCR products are separated by gel electrophoresis, their complementary ends are annealed, and a second PCR reaction in which VL1 and VH4 are used to join these two fragments is performed using the previously annealed products as a template.

a2. Relacement of Linker from ‘Clone B’

The linker separating V_{L} and V_{H} is changed to a linker containing the sequence (Gly₃ Ser)₄ (SEQ. ID. NO:5) by two sequential PCR reactions, using the plasmid with the point mutation corrected as template. The 5′ primer for both sequential reactions is complementary to the vector sequences (M13R; New England Biolabs). The 3′ primer for the first PCR reaction is VL6, and the 3′ primer for the second reaction is VL8. VL6 and VL8 are complementary to the coding strand; the BstXI site in VL8 occurs towards the N-terminus of the V_{H} fragment of UCHT-1. The PCR product resulting from this second PCR reaction encodes the COOH-terminal end of V_{L}, the new linker, and the N-terminus of V_{H} (to just beyond the BSTXI site)

The PCR product from this second PCR reaction is further extended in a third PCR reaction to add the N-terminal region of V_{L}. This reaction uses the second PCR product as the 3′ primer and the M13R (New England Biolabs) primer within the vector as the 5′ primer. The template for this third PCR reaction is the puc18/UCHT-1 ‘Clone B’ plasmid. To substitute the second linker for the first and to attach the PCR product to the remainder of the V_{H}, the PCR product from this third reaction is cut with BamHI which occurs at the junction of V_{L} and the vector and with BstXI which occurs within V_{H}. The puc18/UCHT-1 ‘Clone B’ plasmid also is cut with BamHI and BstXI; the corresponding area was substituted with the new product.

TABLE I.

| | | |
|---|---|---|
| IM-34A: | 5′-GCGGATCCGACATCCAGATGACCCAGACCACC-3′ | (SEQ. ID. NO: 11) |
| | (BamHI site is underlined). | |
| IM-34B: | 5′-CCTCTAGAAGCCCGTTTGATTTCCAGCTTGGT-3′ | (SEQ. ID. NO: 12) |
| | (XbaI site is underlined). | |
| IM-34C: | 5′-CCAAGCTTTCATGAGGAGACGGTGACCGTGGTCCC-3′ | (SEQ. ID. NO: 13) |
| | (HindIII site is underlined). | |
| IM-61: | Coding oligo used for cloning V_{H}: | (SEQ. ID. NO: 14) |
| | 5′-CCGTCGACGAGGTGCAGCTCCAGCAGTCT-3′ | |
| | (SalI site is underlined) | |
| IM-24A: | The coding oligo for the linker is: | (SEQ. ID. NO: 15) |
| | 5′-CTAGAGGAGGTAGTGGAGGCTCAGGAGGTTCTGGAGGTAGTG-3′ | |
| | (partial XbaI and SalI I sites are underlined) | |
| IM-24B: | The corresponding non-coding oligo for the | (SEQ. ID. NO: 16) |
| | linker is: | |
| | 5′-TCGACACTACCTCCAGAACCTCCTGAGCCTCCACTACCTCCT-3′ | |
| | (The corresponding partial SalI and XbaI sites | |
| | are underlined.) | |
| VL1: | 5′end of V_{L} at nt 102-124: | (SEQ. ID. NO: 17) |
| | 5′-CTGGTATCAACAGAAACCAGATC-3′ | |
| VL2: | 3′primer with the correct T at nt #293: | (SEQ. ID. NO: 18) |
| | 5′-GGTGCCTCCAGCGAACGTCCACGGAAG-3′ | |
| VL3: | 5′primer with correct T at nt 293: | (SEQ. ID. NO: 19) |
| | 5′-CTTCCGTGGACGTTCGCTGGAGGCACC-3′ | |
| VH4: | non-coding primer: | (SEQ. ID. NO: 20) |
| | 5′-CTCTGCTTCACCCAGTTCATG-3′ | |
| VL6: | 5′-GCCACCGCTGCCTCCACCTGATCCACCGCCACTACCGCCTCC | (SEQ. ID. NO: 21) |
| | AGCCCGTTTGATTTCCAGCTTGGT-3′ | |
| VL8: | 5′-TCAGGTCCAGACTGCTGGAGCTGCACCTCAGATCCGCCACCGC | (SEQ. ID. NO: 22) |
| | TGCCTCCACCTGAT-3′ | |
| | (BstXI site is underlined) | |

(b) Cloning of PE38.

The cloning of PE38 is described by Benhar et al., Bioconjugate Chem., Vol. 5, No. 4 (1994), and see also U.S. Pat. Nos. 5,981,726 and 5,990,296, incorporated by reference.

(c) Preparation of Immunotoxin Fusion.

The new scFv is cloned into the pET15b E. coli expression vector (Novagen). Sites are first added to the scFv using PCR to make this fragment compatible with the pET15b cloning vector and with the HindIII site from the P. exotoxin-containing plasmid, pRB391 (kind gift of I. Pastan). (Alternatively, the DNA sequence encoding the PE38 fragment can be reconstructed from the pJH8 plasmid which is deposited in the ATCC as ATCC #67208 using standard PCR methods and appropriate oligonucleotide primers. In this method, the pJH8 plasmid would require mutagenesis by PCR to add the HindIII site and the connector sequence present in the pRB391 plasmid and as described in Benhar, et al., 1994, supra. In addition, removal of the 16 amino acids (365-380 of native PE) of domain Ib internal to the PE40 fragment can be accomplished by PCR, resulting in a plasmid which is functionally identical to the PE38 fragment of pRB391. Confirmation that the resulting plasmid is in the same translational frame can be obtained by DNA sequence analysis.)

The amino-terminal residues Met and Ala, encoded by an NcoI restriction site, are added to facilitate expression from the plasmid.

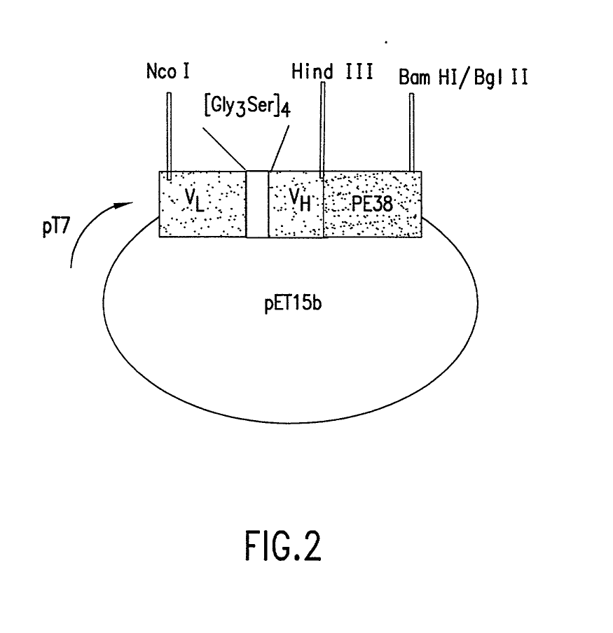

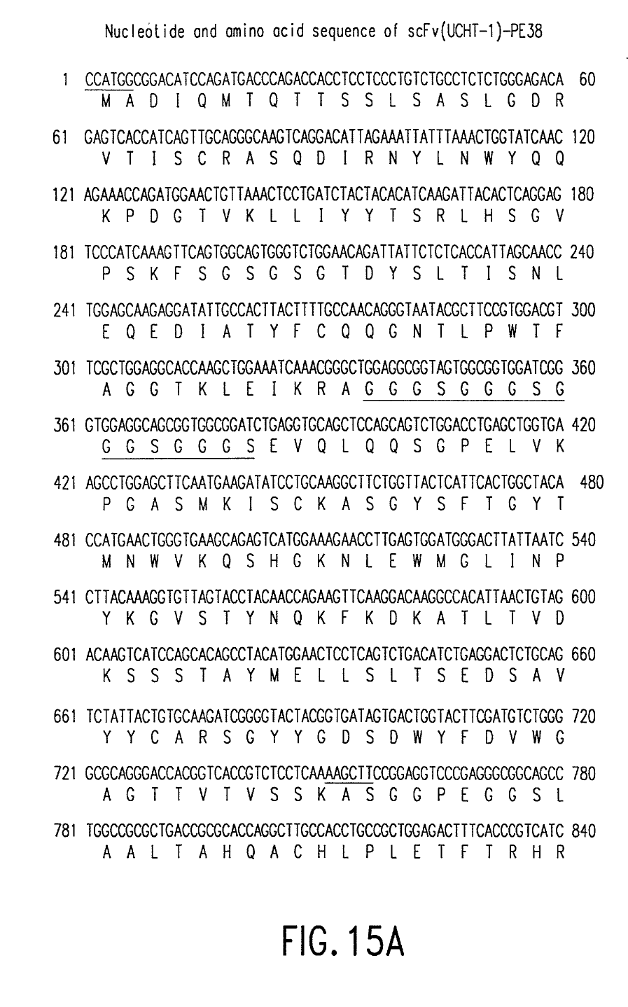

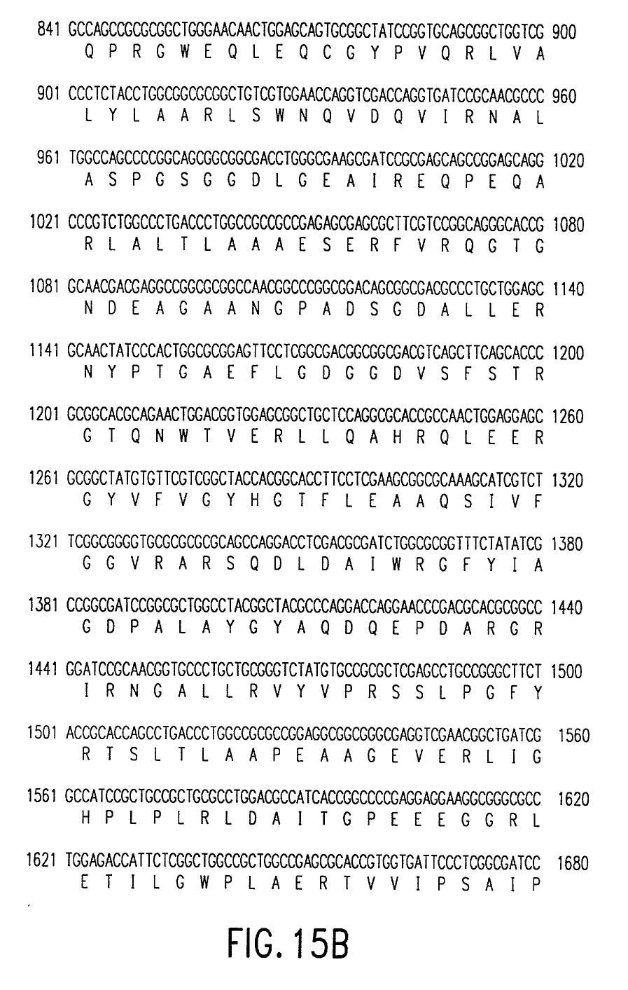

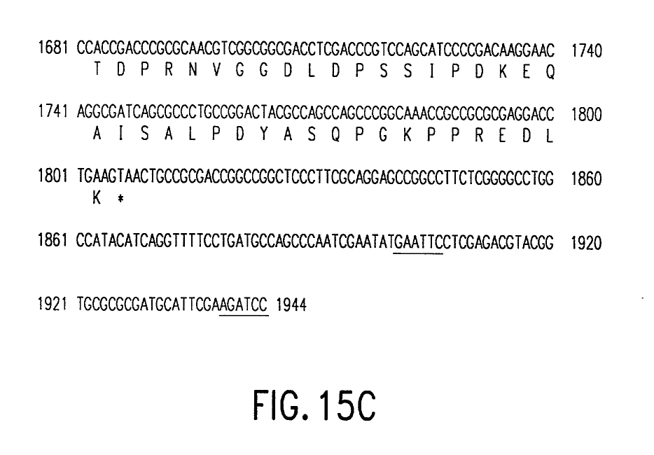

The amino acid and nucleotide sequences of the product (containing Met-Ala at the N-terminus) are given in SEQ. ID NOS:1 and 2, respectively, and FIG. 15. A schematic representation of the protein is shown in FIG. 2.

In SEQ. ID NO:1, V_{L} comprises residues 3-111, the peptide linker occupies residues 112-127, V_{H} comprises residues 128-249, the connector is located at residues 250-254 and truncated PE comprises residues 255-601.

In SEQ. ID. NO:2, DNA sequence encoding the NcoI, HindIII, and the EcoRI restriction sites used for subcloning, and the flexible linker separating the V_{L} from the V_{H} domains, are marked. The 3′-untranslated region, containing the EcoRI site (gaattc), and the BamHI/BglII sites, is deleted.

Expression of scFv(UCHT-1)-PE38 in E. coli strain BLR(DE3) is found to yield a highly homogenous product (i.e. 95% purity or greater) comprising the alanine-led polypeptide having residues 2-601 of SEQ. ID NO:1.

(d) Fermentation, Refolding and Purification of scFv(UCHT-1)-PE38.

A process for the production of recombinant scFv(UCHT-1)-PE38 is established at the 50L scale. PET15b is transformed into E. coli BLR(DE3) (Novagen, Inc.). A fed-batch system using a self-regulatory, pH-stat-glycerol feeding strategy is employed. Feeding starts exactly after the initial amount of carbon source is depleted and glycerol is automatically fed in a limited manner, controlled by the pH. This procedure avoids the detrimental effect of an excess of glycerol and also of complete carbon-source depletion.

The optimal medium contains: KH₂PO₄@6 g/L, KCL@0.6 g/L, MgSO₄x 7H₂O@0.2 g/L, N-Z-Amine A@24.0 g/L, Yeast extract@72 g/L, Fe(III)-ammonium citrate@100 mg/L, MnSO₄x H₂O@12 mg/L and glycerol@10g/L. For optimal expression levels, a lactose pulse induction is needed at OD₅₅₀ of 50. Using this approach, 4.3 kg of wet cell pellet containing 1 kg inclusion bodies are harvested after 24 hours from the fermentation experiment run under the conditions described in Table II (below).

TABLE II
Fermentation Conditions

| Parameter | Conditions |
|---|---|
| Volume | 50 liter |
| Mixing: | 200-250 rpm |
| Aeration/pressure | 1vvm/1 bar |
| PO₂ - control | Manual adjustment |
| pH-control | 6.7< × <7.1 |
| | alkaline: 2 N NaOH |
| Temperature | 37° C. |
| Inocculum | 1.0 L of pre culture grown in LB to |
| | OD₅₅₀ = 1.8 |
| Induction | 50 g/L D-Lactose at OD550 = 52 |
| Harvest: | 11 hours after induction |

Expression levels of 25% of total cellular protein are reached after induction with an excess of D-Lactose at OD₅₅₀ of 50 as assessed by densitomitry of SDS-PAGE gels. Using this approach a productivity of 86 g wet cell pellet (wcp) and 20 g inclusion bodies (IBs) per liter fermenter broth are measured. A product titer of 1.4 g/L is determined by SDS-PAGE and densitometric quantification of scFv(UCHT-1)-PE38.

The scFv(UCHT-1)-PE38 fusion protein is then extracted and refolded according to the general method of Buchner et al. (1992), supra, modified as follows:

(1) Frozen bacterial pellets (65 g), containing induced scFv(UCHT-1)-PE38 in the form of inclusion bodies, are thawed at room temperature and subsequently transferred into 250 ml bottles. 180 ml of TES(50 mM Tris-HCL, pH 7.4, 20 mM EDTA and 100 mM NaCl in water) are added to the bottles and the pellets are thoroughly suspended using a Polytron tissue disrupter. Portions of the suspended cells (30 ml) are distributed to fresh 250 ml bottles and diluted to 180 ml per bottle with TES. 8 ml of lysozyme solution (8 mg/ml in TES) are added to each bottle, the pellets are resuspended, and the suspensions are incubated at room temperature for one hour.

(2) 20 ml of 25% Triton-X100 are added to each bottle, and the mixtures are shaken well. The mixtures are incubated at room temperature for thirty minutes. The cell lysates are then centrifuged at 13,000 rpm for fifty minutes using a GSA rotor.

(3) The pellets are resuspended in 180 ml of TE (50 mM Tris-HCl, pH 7.4, and 20 mM EDTA). The suspensions are homogenized using a Polytron tissue disrupter for two minutes. 20 ml of 25% Triton -X100 are added to each bottle and the mixtures are shaken well. The mixtures are centrifuged at 13,000 rpm for ten (10) minutes.

(4) The detergent (Triton-x100) wash steps described in (b) are repeated three times to produce relatively pure inclusion bodies. The inclusion bodies are resuspended in 180 ml of TE, and are then centrifuged at 13,000 rpm for ten (10) minutes.

(5) The TE rinse steps described in (3) are repeated three times. The inclusion bodies are pooled and frozen as pellets at −70° C.

(6) 42 ml of solubilization buffer containing 6M Guanidine-HCl (MW=95.53) with 0.1 M Tris -HCl, pH 8.0 and 2 mM EDTA, is added to pooled inclusion bodies. The inclusion bodies are suspended by pipette. The suspension is transferred to two 50ml centrifuge tubes. The contents are incubated at room temperature overnight, and centrifuged.

(7) 100 mg batches of denatured inclusion body protein are processed by reduction and renaturation. Dithioerythritol (DTE) is added to 0.3 M and the mixture is incubated at room temperature for two hours prior to the rapid addition of this sample (100 mg denatured inclusion body protein) to 100 volumes of refolding buffer. The refolding buffer is prepared by combining 0.1M Tris, pH 8.0, 0.5 M L-arginine-HCl (FW 210.7 g), and 2mM EDTA, adjusted to pH 9.5 with 10N NaOH, and equilibrated to 8-10° C. prior to the addition of oxidized glutathione (GSSG, MW 612.6g) to 8 mM. The sample is allowed to refold at 10° C. for 30-40 hours without agitation. The sample is concentrated in a biocentrator and dialyzed into 20 mM Tris-HCl, pH 7.4, 1 mM EDTA and 100 mM Urea.

(8) Refolded immunotoxin is purified by two sequential rounds of anion exchange chromatography, the first using Fast-Flow Q (Pharmacia) with a salt step gradient elution, and the second, using a Q5 column (BioRad)followed by a salt gradient elution. The following buffers are used during column chromatography for step and linear gradient elutions:

equilibration: 20 mM Tris-HCl, pH 7.4, 1 mM EDTA

wash: 20 mM Tris-HCl, pH 7.4, 1 mM EDTA, 0.08 M NaCl

elution: 20 mM Tris-HCl, pH 7.4, 1 mM EDTA, 0.28 M NaCl

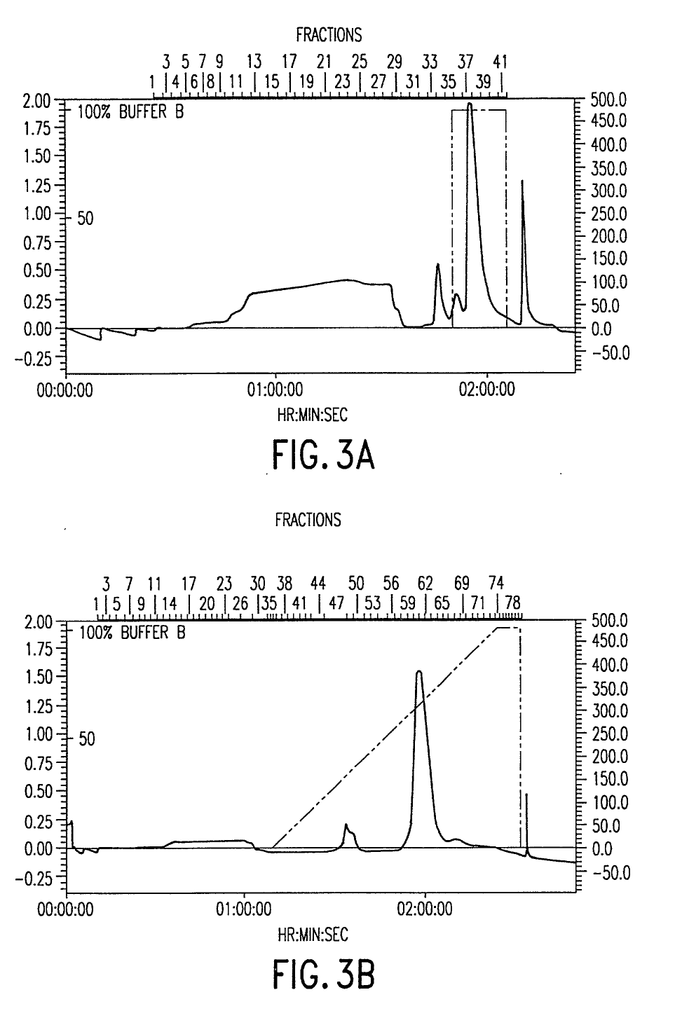

FIG. 3A shows a typical Fast Flow Q column purification profile. The eluted peak is then diluted 5-fold with equilibration buffer and applied to the Q5 column in the subsequent purification step. FIG. 3B shows a typical Q5 column profile.

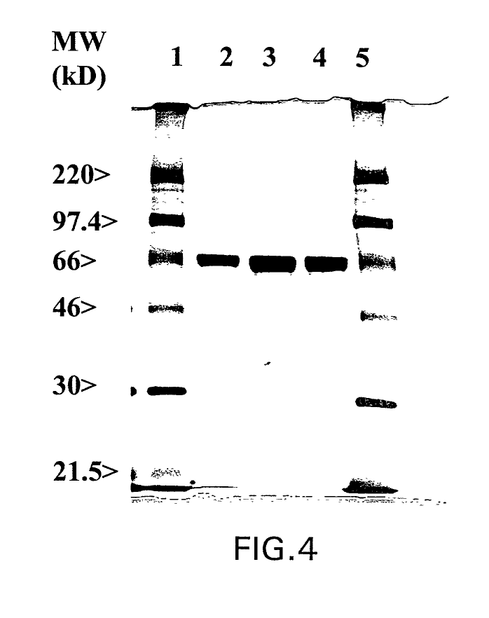

A single peak is recovered from the second anion-exchange column (FIG. 3B). This peak correlates with scFv(UCHT-1)-PE38 (>95% pure) as evidenced by mobility at the expected position (64.5 kD) following SDS-PAGE (FIG. 4) and by cross-reaction on Western blots probed with rabbit anti-PE38 polyclonal antibodies (not shown in figures).

The yield of correctly refolded scFv(UCHT-1)-PE38 recovered using the above procedure has reached 50 mg/L using the above-indicated concentrations of DTE and GSSG.

The refolding protocol is reproduced in sixteen batches of material, which are refolded to yield material with very similar IC₅₀ values as determined in the MTS assay (Table III).

The first eleven batches produce a protein which has a point mutation which converts serine to arginine at residue 63 in the third framework region of the variable light chain of UCHT-1. Based on the in vitro results presented on Table III infra, this mutation appears to have little or no consequence in terms of the specific in vitro cytotoxicity.

Five batches of protein (i.e. batches 12, 13, 14, 15, and 16), in which the point mutation is corrected, are refolded.

Due to the high reproducibility in the MTS assay, batches 12 and 13, and batches 14, 15 and 16, are pooled. The pooled batches are tested for potency in the MTS assay (see Table III) and then themselves combined to form “Pooled Batches 12-16”, used in the majority of the in vitro studies, and in the in vivo studies, reported herein. Pooled Batches 10A-12A, also comprising the corrected material, are similarly obtained and tested (see Table III).

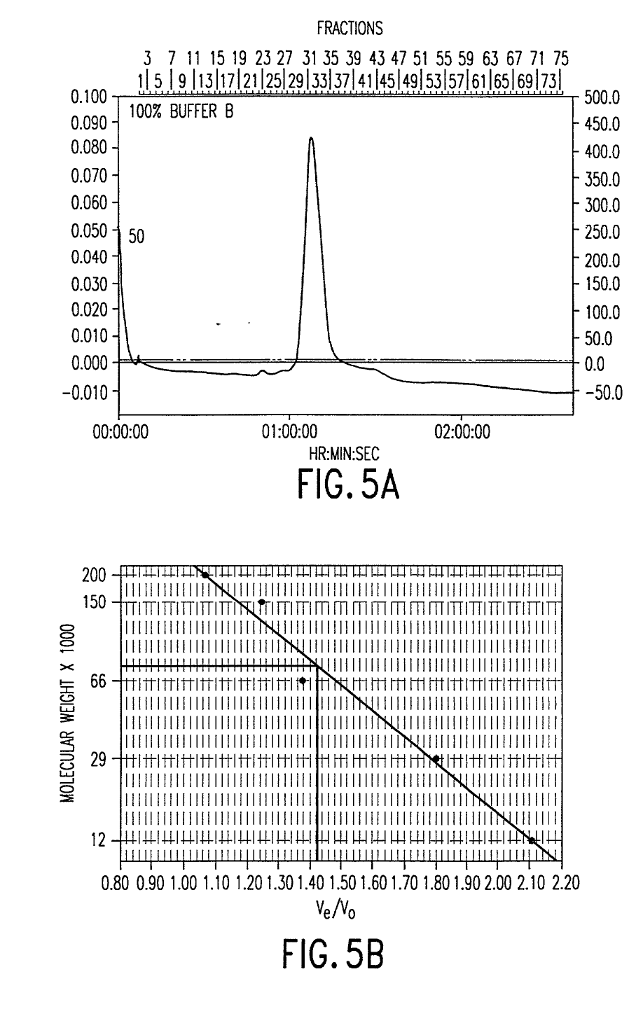

Analysis by non-denaturing PAGE reveals that purified scFv(UCHT-1)-PE38 exists in solution as a monomer (not shown in figures). In addition, there appears to be no aggregated material, as assayed by size exclusion column chromatography (Sephacryl S200) (FIG. 5A (sample) and SB (marker))or by dynamic light scattering (not shown). Essentially all of the protein migrates near the position of bovine serum albumin (66 kD).

### Biological Activity of Immunotoxins

(1) MTS Assay of scFv(UCHT-1)-PE38.

Specific toxicity towards a CD3⁺-expressing human Jurkat T-cell line is demonstrated using an MTS assay three days after addition of immunotoxin to cells.

In the MTS assay, cell viability is measured by adding MTS, i.e. (3(4,5-dimethythiazol-2-yl)-5-(3-carboxymethoxy-phenyl)-2H-tetrazolium, inner salt), which is metabolized by viable cells in the presence of the electron coupling agent, phenazine methosulfate, to a water-soluble formazan derivative. The absorbance at 490 nm of the formazan derivative is proportional to the number of viable cells. The number of viable cells at the time of test compound addition is compared to the number of viable cells present at 72 hours post-compound addition. The negative control for non-specific toxicity is the human CD3⁻ Ramos B-cell line.

The IC₅₀ and standard deviations of 16 samples of refolded protein on Jurkat and Ramos are reported on Table III.

TABLE III
Toxicity of different refolded batches on human
CD3⁺ (Jurkat) or CD3⁻ (Ramos) cell lines produced using
either a point variant of scFv(UCHT-1)-PE38 (Batches 1-11
and Pooled Batches 4-7 and 8-11) or scFv(UCHT-1)-PE38
(Pooled Batches 12-13; 14-16; 12-16; and 10A-12A).

| | | Jurkat (CD3⁺) | | Ramos (CD3⁻) | |
|---|---|---|---|---|---|
| | Mean | Std. | | | |
| | IC₅₀ | Error | | Mean IC₅₀ | |
| Batch(es) | (ng/ml) | of Mean | N | (ng/ml) | n |

Point variant

| | | | | | |
|---|---|---|---|---|---|
| 1 | 1.51 | 0.37 | 9 | >10 | 2 |
| | | | | >25 | 4 |
| 3 | 1.03 | 0.17 | 5 | >25 | 1 |
| | | | | >250 | 1 |
| 4 | 0.75 | 0.10 | 5 | >25 | 1 |
| | | | | >250 | 1 |
| | | | | >10,000 | 1 |
| 5 | 0.57 | | 1 | >10,000 | 1 |
| 6 | 0.18 | | 1 | >10,000 | 1 |
| 7 | 0.27 | | 1 | >10,000 | 1 |
| 8 | 0.18 | | 2 | >25 | 2 |
| 9 | 0.22 | 0.04 | 3 | >25 | 2 |
| | | | | >1000 | 1 |
| 10 | 0.21 | | 1 | >25 | 1 |
| 11 | 0.26 | | 1 | >25 | 1 |
| Pooled 4-7 | 0.26 | 0.04 | 5 | >25 | 3 |
| Pooled 8-11 | 0.27 | 0.07 | 3 | >25 | 1 |

scFv(UCHT-1)-PE38

| | | | | | |
|---|---|---|---|---|---|
| Pooled 12-13 | 0.18 | | 1 | >25 | 1 |
| Pooled 14-16 | 0.28 | | 1 | >25 | 1 |
| Pooled 12-16 | 0.63 | 0.15 | 16 | >25 | 8 |
| | | | | >10,000 | 4 |
| Pooled 10A- | 1.3 | 0.30 | 7 | >100,000 | 2 |
| 12A | | | | | |

The scFv(UCHT-1)-PE38 immunotoxin is very potent (≈10 pM) as measured by CD3⁺ cell killing in the MTS assay. At high concentrations, the protein reduces the viable cell number below the starting cell number, and therefore behaves as a cytotoxic agent.

(2) Thermal Stability of scFv.

The thermal stability of scFv (UCHT-1)-PE38 is measured using the MTS assay described above. Samples are incubated at 4° C., 25° C. and 37° C. at 100 μg/ml in PBS. As is evident from Table IV, the material is completely stable at 4° C. and 25° C. for one month. At 37° C., there may be a slight increase in the IC₅₀ at 21 or 28 days.

TABLE IV
Thermal stability of scFv(UCHT-1)-PE38.
IC₅₀ + std. Dev. (ng/ml)

| Time (days) | 4° C. | 25° C. | 37° C. |
|---|---|---|---|
| 0 | 2.0 ± 0.5 | — | — |
| 7 | 1.6 ± 1.1 | 0.8 ± 0.1 | 1.9 ± 0.8 |
| 14 | 1.2 ± 0.8 | 1.4 ± 0.4 | 2.1 ± 1.2 |
| 21 | 2.3 ± 2.5 | 1.6 ± 0.4 | 1.6 ± 0.9 |
| 28 | 2.4 ± 1.0 | 1.5 ± 0.8 | 3.2 ± 1.8 |

(3) Protein Synthesis Inhibition Assay for scFv(UCHT-1)-PE38.

Cells are incubated overnight in the presence or absence of immunotoxin. The next morning, cells are pulsed for three hours with ³H-leucine. The plates are frozen at −80° C. for cell lysis, and then harvested onto a glass filter fibermat using a cell harvestor and extensive water washes. Incorporation into protein is measured using a Wallac Betaplate reader. Typically, in the absence of immunotoxin, ³H-leucine incorporation is 3,000-4,000 cpm; background from label added immediately prior to cell processing is 400-700 cpm. The standard deviation of triplicate wells within one plate is generally <10%, and variation of the mean incorporation between plates is <10%.

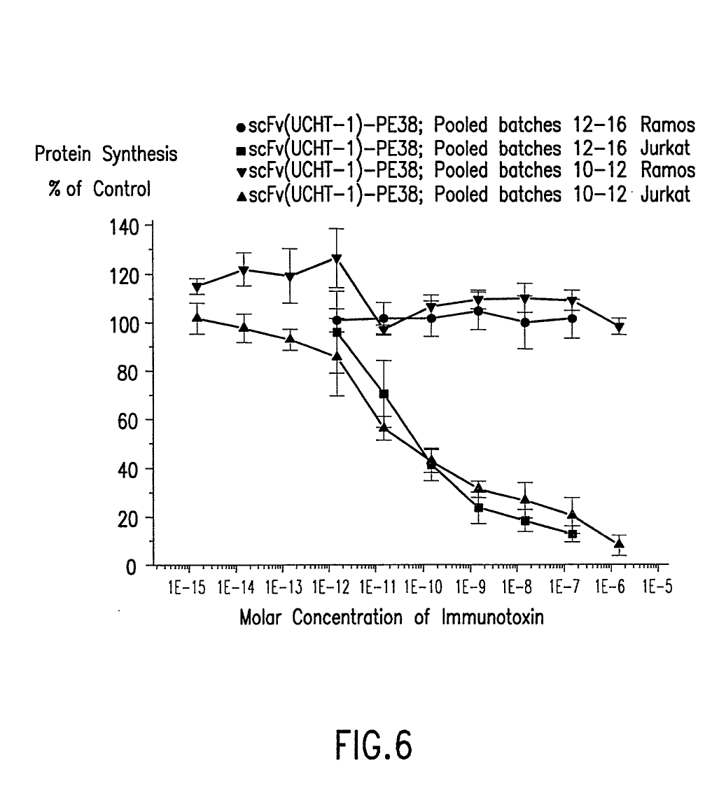

In FIG. 6, protein synthesis inhibition in Jurkat (CD3⁺) and Ramos (CD3⁻) cells by Pooled Batches 12-16, or Pooled Batches 10A-12A, of scFv(UCHT-1)-PE38 is shown. The plot shows the mean and standard error of the mean for nine determinations for pooled Batches 12-16, and for three determinations for Pooled Batches 10A-12A. The IC₅₀ of the scFv(UCHT-1)-PE38 in this assay is 6.7±1.9 ng/ml or 104±29 pM.

The curves appear similar from both batches, and the selectivity for killing is present even at the highest concentration tested (100 μg/ml). At the higher concentrations, the number of cells is reduced below the starting cell number.

FIG. 6 also shows the selectivity of toxicity for the CD3⁺ Jurkat cell line; an IC₅₀ for killing CD3⁻ Ramos cells is not attained in these experiments even with with 4 or 5-logs higher concentration of scFv(UCHT-1)-PE38.

(4) Human Blood Mixed Lymphocyte Reaction (MLR).

The ability of the scFv(UCHT-1)-PE38 immunotoxin to prevent proliferation of alloreactive human peripheral blood mononuclear cells (PBMC) is measured using a two-way mixed lymphocyte reaction (MLR). The MLR is a measure of allo-stimulation. Interference with cell proliferation in the MLR assay is a measure of the potency of an immunosuppressive agent to act upon intact human blood cells.

The human MLR is performed according to standard procedures. PBMC from three different donors (A, B, C) are isolated on Ficoll from buffy coats with unknown HLA type (Kantonspital/Basel/Blutspendez-entrum). Cells are kept at 2×10⁷ cells/1 ml (90% FCS, 10% DMSO) in cryotubes (Nunc) in liquid nitrogen until use. To initiate the MLR, the cells are thawed, washed and counted.

In each of two experiments (“A” and “B”), 3 individual, 2-way reactions (A⇄B, A⇄C, B⇄C) are established by mixing cells from 2 different donors in a ratio of 1:1 by cell number. The mixed cells (total 4×10⁵ cells/0.2 ml) are co-cultured in triplicate for 6 days at 37° C., 5% CO₂. Cyclosporine A serves as a positive control.

Cultures are performed in the presence of increasing concentrations of immunotoxin (Pooled Batches 12-16) or control.

Proliferation is determined by ³H-TdR uptake (1 mCi/0.2ml) over the last 16 hours of culture.

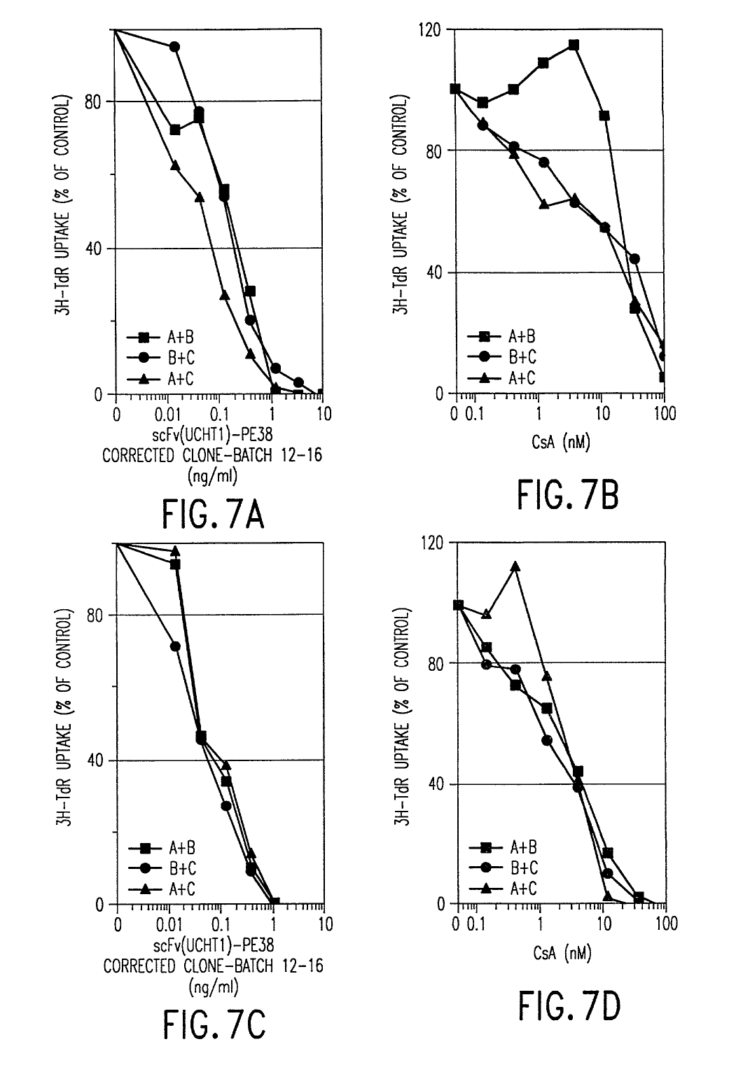

The results are presented on Table V and shown graphically on FIG. 7.

TABLE V
Inhibition of human mixed lymphocyte reactions by
scFv(UCHT-1)-PE38 compared to cyclosporine A
in two experiments, (I) and (II).

| | | | | |
|---|---|---|---|---|
| | | | | Mean ± |
| | | | | Std. |
| Compound | A <−> B | B <−> C | B <−> C | Dev. |

Experiment A

| | | | | |
|---|---|---|---|---|
| scFv(UCHT-1)-PE38 | 0.15 | 0.13 ng/ml | 0.05 | 0.11 ± |
| | ng/ml | | ng/ml | 0.053 |
| | | | | ng/ml |
| Cyclosporine A | 22.9 nM | 17.3 nM | 14.4 nM | 18.2 ± |
| | | | | 4.32 nM |
| Experiment B | | | | |
| scFv(UCHT-1)-PE38 | 0.036 | 0.033 | 0.036 | 0.035 ± |
| | ng/ml | ng/ml | ng/ml | 0.002 |
| | | | | ng/ml |
| Cyclosporine A | 2.6 nM | 1.6 nM | 2.6 nM | 2.27 ± |
| | | | | 0.58 nM |

The potency of scFv(UCHT-1)-PE38 in preventing proliferation of human blood PBMC in an in vitro mixed lymphocyte reaction (MLR) in the above two experiments is determined to be 0.11±0.053 ng/ml and 0.035±0.002 ng/ml, resulting in a global IC₅₀ of 0.072±0.053 ng/ml (1.12 pM).

The data demonstrate that scFv(UCHT-1)-PE38 efficiently suppresses allo-specific T cell activation in human MLR.

(5) Inhibition of Human CD3ε Transgenic Murine Splenocyte Concanvalin A-Stimulated Proliferation by scFv(UCHT-1)-PE38.

Human CD3ε transgenic mice: A strain of human CD3ε transgenic mice is obtained from C. Terhorst (Beth Israel Deaconess Medical Center). The phenotype of transgenic mice expressing high and low copy numbers of human CD3ε is described by Wang et al. (1994) PNAS 91: 9402. Mice which express high copy numbers of the transgenic human CD3E gene have no T or NK cells even when heterozygous, and thus have a knockout phenotype. The tgε600 strain reportedly has ˜3 copies of the human CD3ε transgene integrated chromosomally at an unknown location. Homozygous, low-copy number transgenic mice such as tgε600 mice express only a limited number of T cells. In contrast, when heterozygous for tge600, mice have near normal numbers of T cells most of which express both human and murine CD3ε.

The genetic background of these mice is mixed; the transgene being introduced by pronuclear injection of F2 embryos from a CBA by C57BL/6 cross, and therefore, siblings are genetically different.

The transgenic mice homozygous for human CD3ε are bred at Charles River Laboratories with C57BL/6 wildtype mice to generate heterozygous mice.

The animals are maintained as homozygotes for the transgene and used as heterozygotes after back-crossing to C57BL/6.

Animals heterozygous for the tgε600 insertion are used for testing in vitro sensitivity to scFv(UCHT-1)-PE38 and in vivo depletion caused by scFv(UCHT-1)-PE38 after intravenous or intraperitoneal administration. Pooled Batch 12-16 was used for these experiments. For the in vitro work, Fl progeny of a CBA x C57BL/6 cross are used as control animals. In the in vivo experiments, untreated heterozygous tge600 mice serve as a control group.

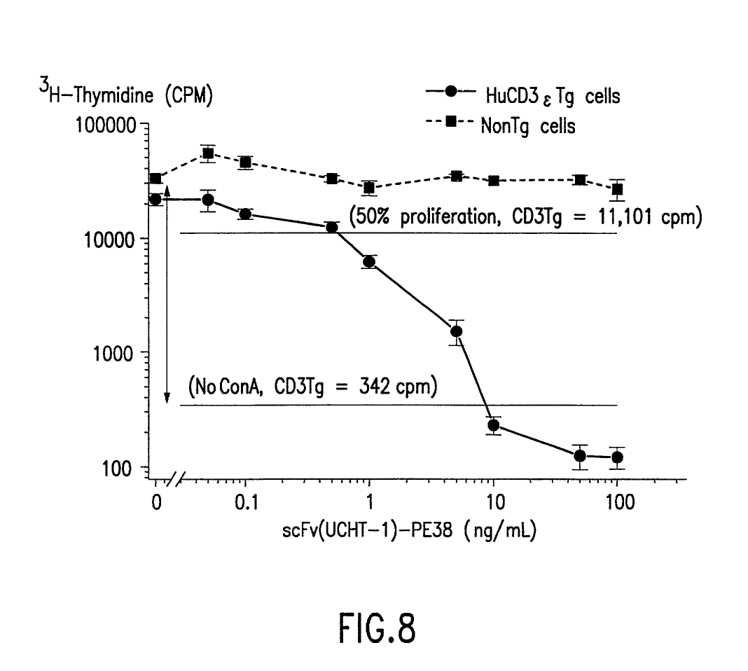

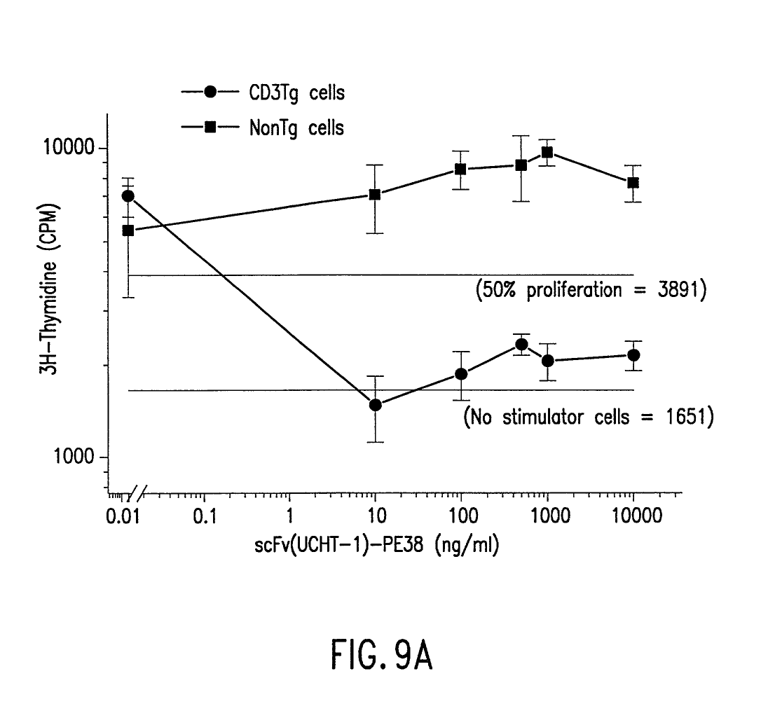

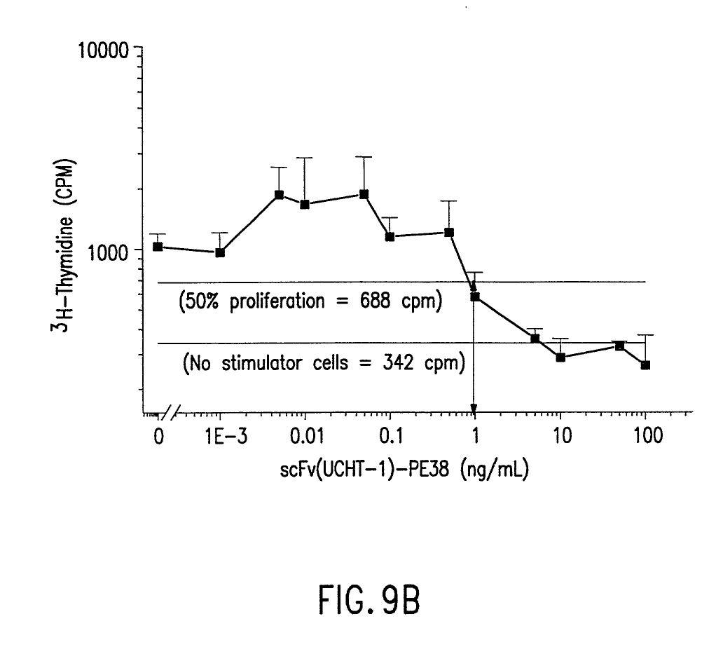

The ability of scFv(UCHT-1)-PE38 to inhibit in vitro proliferation of splenocytes from transgenic mice expressing human CD3ε is assessed by Concanavalin A-induced proliferation (FIG. 8) as well as a one-way mixed lymphocyte reaction (FIG. 9).

The spleens are disrupted, passed through a nylon filter (0.45 μm), and gently pipetted with a 1 ml syringe to generate a single cell suspension. Red blood cells are lysed using ACK buffer (0.15 M ammonium chloride, 1 mM potassium carbonate, 0.1 mM EDTA), and the resulting suspension washed three times into RPMI-1640 supplemented with 5% FBS. Concanavalin A (Sigma) is added to the wells at 5 ug/ml. The plates are incubated for three days at 37° C. in 5% CO₂. On the third day, 1 uCi/well of ³H-thymidine is added. After 24 hours the cells are harvested onto glass fiber filters, and the ³H-thymidine incorporation measured using a Wallac beta plate reader.

As shown in FIG. 8, addition of scFv(UCHT-1)-PE38 blocks Con A (5 ug/ml)-induced proliferation of human CD33ε transgenic (“HuCD3εTg”) splenocytes, but not proliferation of non-transgenic, B6CBAF1 (“NonTg”) splenocytes. Dose-dependent inhibition of the cells from the transgenic mice is observed with a calculated IC₅₀ of 0.6 ng/ml. This is in good agreement with cytotoxicity against Jurkat cells (0.63±0.15 ng/ml). At high concentrations, >100% inhibition is observed (i.e. less proliferation than observed in the absence of ConA), suggesting that all ConA-responsive splenocytes are sensitive to scFv(UCHT-1)-PE38. The line labelled “No ConA” represents the proliferative response in the absence of ConA, due to media alone.

(6) Inhibition of Proliferation of Human CD3ε Transgenic Murine Splenocytes by scFv(UCHT-1)-PE38 in One-Way MLR.

The ability of scFv(UCHT-1)-PE38 to inhibit human CD3ε splenocyte T cell proliferation in vitro is assessed using a one-way mixed lymphocyte reaction. In a one-way MLR, proliferation is due to direct recognition of allo-MHC II by allo-reactive huCD3ε transgenic murine splenocytes. Not all T cells are allo-reactive, resulting in a smaller percentage of responding transgenic splenocytes, consistent with the reduced signal to noise of the assay and the increased variability between experiments.

HuCD3ε transgenic splenocytes (“CD3Tg cells”) are prepared as in section 5 above. Spleen cells of non-transgenic B6CBAF1 mice (“NonTg cells”) are used as a control.

A single cell suspension of Balb/C splenocytes prepared as in section 5 above is treated with mitomycin C (30 μg/ml) for 20 min at 37° C., and washed into MLR media.

The mitomycin C-treated BALB/c stimulator cells are added to flat-well Corning 96-well plates at 4×10⁵ cells/ml. Splenocytes from the transgenic mice are added to the wells at 2×10⁵ cells/ml, and the plates incubated for three days at 37° C. in 5% CO₂. On the third day, 1 μCi/well of ³H-thymidine is added. After 16 hours, the cells are harvested onto glass fiber filters, and ³H-thymidine incorporation measured using a Wallac beta plate reader.

As shown in FIGS. 9A and 9B, the scFv(UCHT1)-PE38 immunotoxin inhibits the allogeneic MLR response in cultures containing huCD3ε Tg splenocytes, but not non-transgenic control splenocytes. Dose-dependent inhibition of the cells from the transgenic mice is observed, with a calculated IC₅₀ of 0.6 ng/ml. At high concentrations, >100% inhibition is observed, suggesting that all allo-reactive huCD3ε T cells are sensitive to scFv(UCHT-1)-PE38. The MLR response between non-transgenic B6CBAF1 spleen cells and mitomycin C treated Balb/C (APC) splenocytes is not inhibited by scFv(UCHT-1)-PE38 (FIG. 9A).

Accordingly, the immunotoxin is found to inhibit a MLR response of huCD3ε transgenic splenic (T-cells) cells stimulated by fully allogeneic mitomycin C-treated BALB/C splenic (APC) cells, in a dose-dependent manner.

The potency of the immunotoxin in this assay is ˜0.9 ng/ml, i.e., ˜14 pM.

(7) Jurkat Hollow Fiber Implant Model

Eight hollow fibers are implanted into a single nude mouse: four are placed intraperitoneally, and another four are placed subcutaneously. two of the four hollow fibers in each location contain CD3⁺ Jurkat cells; one of the four fibers in each location contains LS174T colon carcinoma cells; and one contains MDA-MB-435S breast carcinoma cells. Six animals comprise a group.

It is noted that the material used for these studies contains a point mutation from T to G at nucleotide 195 of Seq. ID NO:2 that changes serine (UCHT-1) to arginine (mutant) at residue 65 of SEQ. ID NO:1 (i.e. in the third framework region of the variable light chain). The efficacy of this material in the 3-day MTS assay is equivalent to that of scFv(UCHT-1)-PE38 with no mutation (Table III).

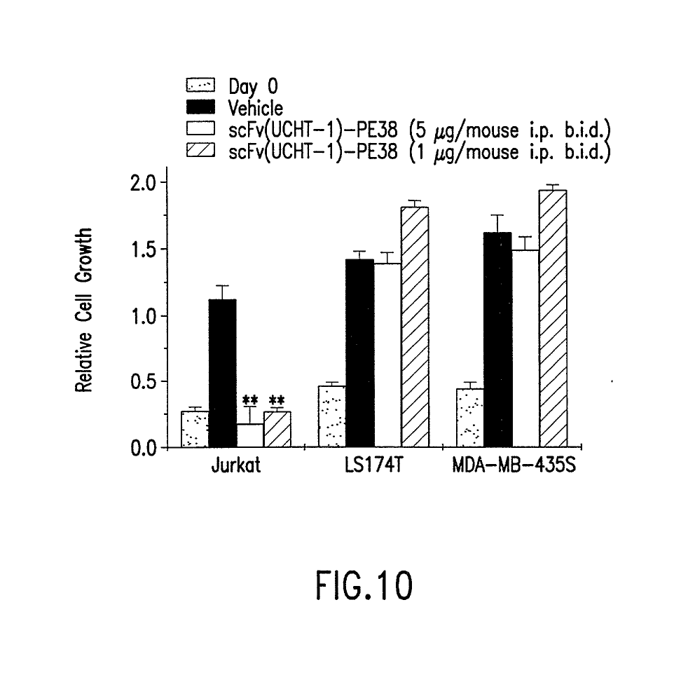

FIG. 10 depicts relative cell growth of Jurkat cells in hollow fibers implanted in the peritoneal cavity in nude mice, following intraperitoneal administration (150 μL in saline vehicle per mouse) of scFv(UCHT-1)-PE38 at a dose level of 1 μg/mouse twice daily or 5 μg/mouse twice daily from days 3-6. The fiber is retrieved on day 10.

Also in this model, approximately 75% inhibition of Jurkat cell growth in intraperitoneally implanted hollow fibers is seen using 1 μg/mouse dosed i.p. (twice daily for 4 days) or using 3 μg/mouse dosed i.v. (twice daily for 4 days).

The immunotoxin is shown to have systemic in vivo efficacy in killing a human T-cell line implanted in nude mice after i.p. or i.v. administration, and the growth inhibition observed is specific for CD3⁺ cells.

(8) T-cell Depletion in Human CD3ε Transgenic Mice.

Tgε600/C57BL6 heterozygous mice described as above are treated with 4 μg/mouse of immunotoxin (Pooled batches 12-16) twice daily for four days. One day following the final treatment, lymph nodes (LN) and spleens are removed, and single cell suspensions are prepared from individual mice.

The percentage of CD3-positive cells is assessed by two-color FACS analysis performed on single cell suspensions using FITC-anti huCD3ε antibodies (to measure expression of human CD3ε and phycoerythrin (PE) conjugated-anti mCD3ε antibodies (500A2-PE) (to measure expression of mouse CD3). The number of T cells in each organ is determined by multiplying the number of total cells recovered from the organ by the percentage of CD3-positive cells.

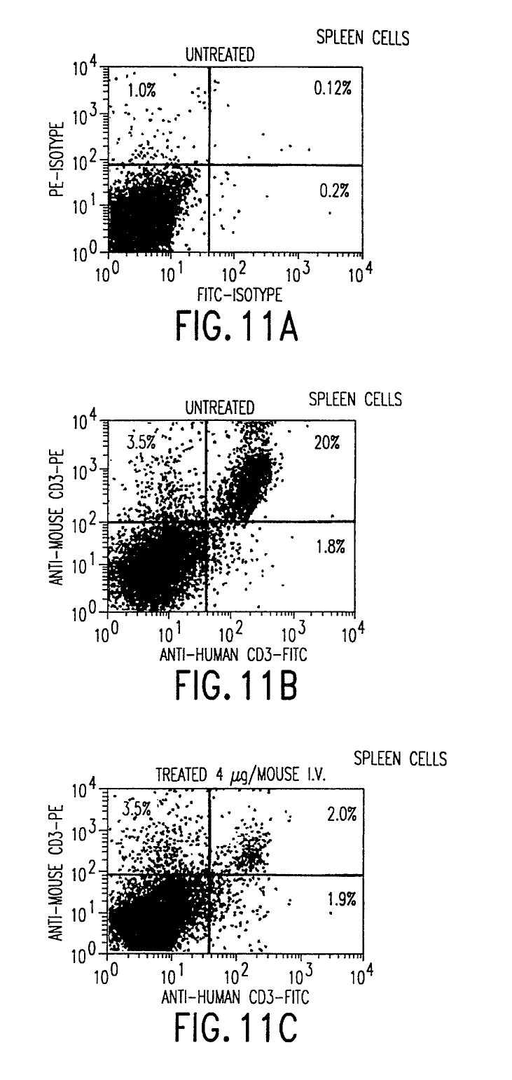

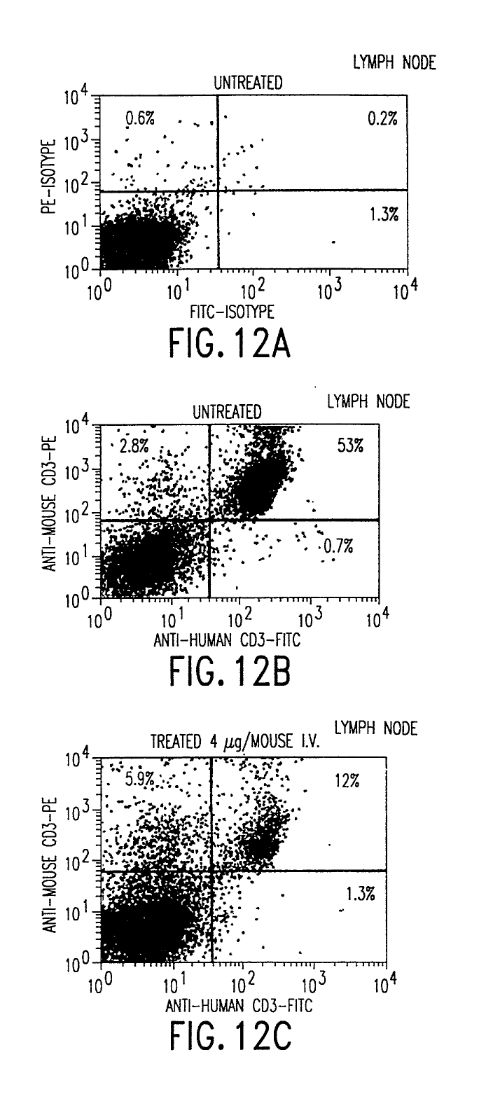

FIGS. 11A,B and C and FIGS. 12A, B and C show representative FACS analyses of the spleen (FIG. 11), and the lymph node (FIG. 12) from treated and untreated animals. Each figure shows three plots as follows: (A) cells from untreated mice stained with control antibodies of identical isotype to the test antibodies; (B) cells from untreated mice double-stained with anti-human and anti-mouse CD3 MAb's; and (C) cells from mice treated with scFv(UCHT-1)-PE38 double-stained with anti-human and anti-mouse CD3 MAb's.

FIG. 11A shows that non-specific staining of cells by isotype matched control antibodies is low. No difference in non-specific staining is seen between treated or untreated mice (data not shown).

FIG. 11B shows that ˜20% of the total cells in the spleen in an untreated transgenic animal are positive for both mCD3 and huCD3 (upper right quadrant). A small percentage of cells express mouse CD3, but do not express human CD3 (3.5%; upper left quadrant).

FIG. 11C shows that systemic treatment with scFv(UCHT-1)-PE38 reduces the percentage of cells that express both huCD3 and mCD3 from about 20% to 2%.

The results of FACS analyses of lymph nodes (LN) from treated and untreated transgenic mice shown in FIG. 12 are similar to the results seen in the FACS analysis of spleen cells from the transgenic mice. That is, non-specific staining of cells by isotype matched control antibodies is low (FIG. 12A). In an untreated transgenic mouse, ˜53% of the total cells in the LN are positive for both mCD3 and huCD3 (upper right quadrant, FIG. 12B). A small percentage of cells express mouse CD3, but do not express human CD3 (2.8%; upper left quadrant). After intravenous administration of scFv(UCHT-1)-PE38 (4 μg/animal) twice daily for four days, the percentage of double positive LN cells that express huCD3 and mCD3 is reduced from ˜53% to 12% (FIG. 12C).

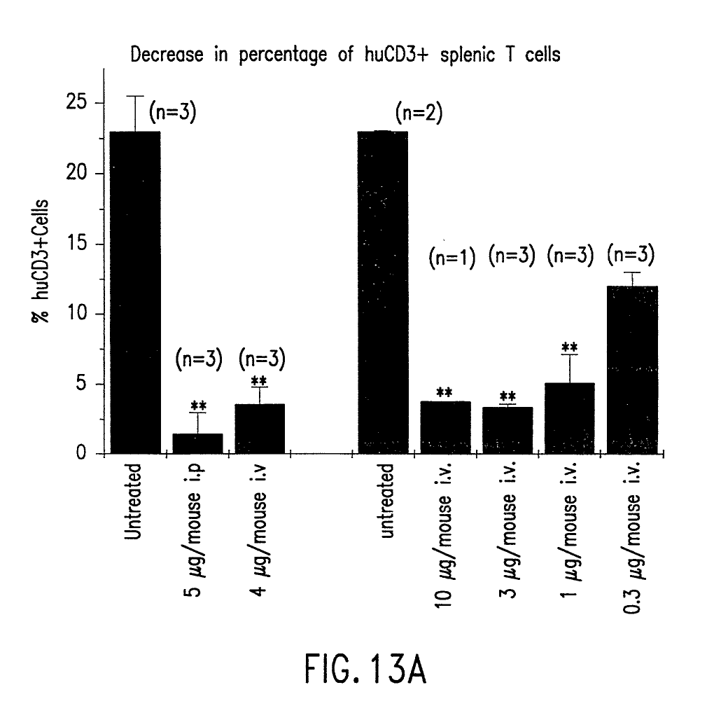

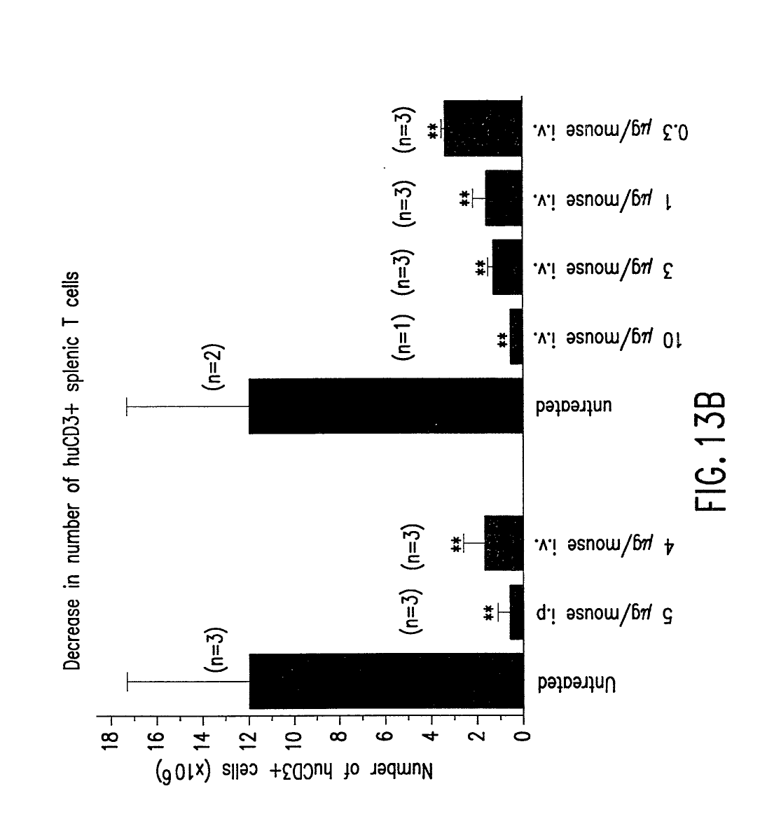

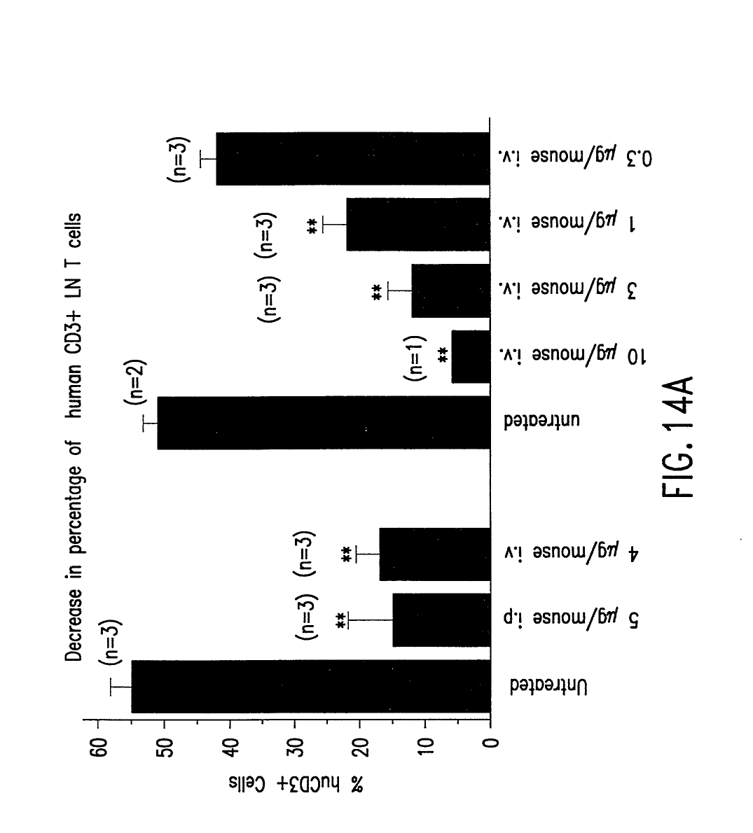

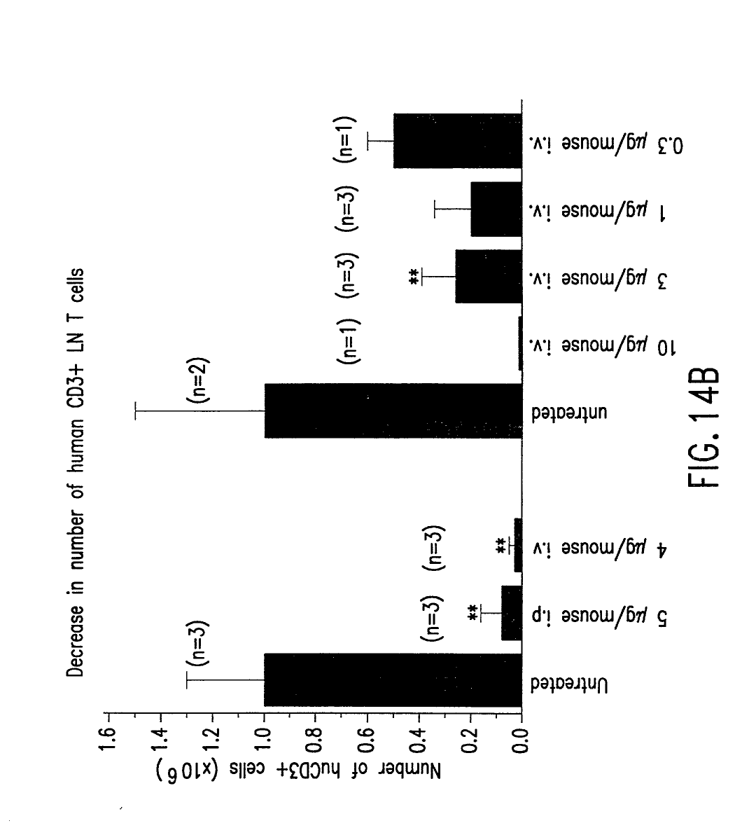

The effect of different dosing regimens on the percentage and number of cells double positive for both mouse and human CD3 is shown for the three tested tissues in FIGS. 13A and B and 14A and B″. Results are similar for both spleen (FIG. 13) and lymph node (FIG. 14). scFv(UCHT-1)-PE38 causes statistically significant depletion of double positive T-cells when administered either i.v. or i.p. in a twice a day dosing regimen. In addition, dose-dependent depletion is observed in both tissues after systemic administration.

Summarizing the data generated, 4 μg/mouse i.v. or 5 μg/mouse i.p. for 4 days b.i.d. result in 86% and 95% depletion in the number of splenic huCD3 T cells recovered. Statistic-ally significant reduction of spleen cell number is seen with 0.3 μg/mouse i.v. b.i.d×4 days and with 1 μg/mouse i.v. b.i.d. when the percentage of huCD3 positive cells is considered. Thus the lowest effective dose appears to be 1 μg b.i.d.×4 days for splenic depletion.

For the lymph node, treatment with 4 μg/mouse i.v. or 5 μg/mouse i.p. for 4 days b.i.d. results in 97% and 92% depletion in the number of huCD3 T cells recovered. Statistically significant reduction of lymph node cell number is seen in mice treated with 3 μg/mouse i.v. b.i.d×4 days and with 1 μg/mouse i.v. b.i.d.×4 days when the percentage of huCD3 positive cells in lymph node is considered. Thus, the lowest effective dose appears to be 1 μg b.i.d.×4 days for lymph node depletion.

What is claimed is:

1. A recombinant immunotoxin polypeptide and pharmaceutically acceptable salts thereof comprising a CD3-binding domain and a Pseudomonas exotoxin (PE) mutant, said PE mutant having ADP-ribosylating and translocation functions but substantially diminished cell-binding ability.

2. A recombinant immunotoxin polypeptide and pharmaceutically acceptable salts thereof according to claim 1 wherein the CD3-binding domain comprises an anti-CD3 antibody or CD3-binding fragment thereof.

3. A recombinant immunotoxin polypeptide polypeptide and pharmaceutically acceptable salts thereof according to claim 2 wherein the anti-CD3 antibody or CD3-binding fragment thereof binds an epitope on the ε chain of human CD3.

4. A recombinant immunotoxin polypeptide and pharmaceutically acceptable salts thereof according to claim 2 wherein the anti-CD3 antibody or CD3-binding fragment thereof binds an epitope formed by the ε and γ chains of human CD3.

5. A recombinant immunotoxin polypeptide and pharmaceutically acceptable salts thereof according to claim 2 wherein the CD3-binding domain comprises a Fab fragment of an anti-CD3 antibody.

6. A recombinant immunotoxin polypeptide and pharmaceutically acceptable salts thereof according to claim 2 wherein the CD3-binding domain comprises the Fv region, or a CD3-binding fragment thereof, of an anti-CD3 antibody.

7. A recombinant immunotoxin polypeptide and pharmaceutically acceptable salts thereof according to claim 2 wherein the CD3-binding domain comprises monoclonal antibody UCHT-1 or a CD3-binding fragment thereof.

8. A recombinant immunotoxin polypeptide polypeptide and pharmaceutically acceptable salts thereof according to claim 2 wherein the CD3-binding domain comprises the Fv region, or a CD3-binding fragment thereof, of an antibody selected from: monoclonal antibody UCHT-1, an antibody having a variable region which is at least 80% identical to the variable region of UCHT-1, an antibody having complementarity-determining regions identical with those of UCHT-1 and having at least one sequence segment of at least five amino acids of human origin, and an antibody competing with UCHT-1 for binding to human CD3 antigen at least about 80% as effectively on a molar basis, and having at least one sequence segment of at least five amino acids of human origin.

9. A recombinant immunotoxin polypeptide and pharmaceutically acceptable salts thereof according to claim 2 wherein the CD3-binding domain comprises a single chain Fv of an anti-CD3 antibody.

10. A recombinant immunotoxin polypeptide and pharmaceutically acceptable salts thereof according to claim 8 wherein the Fv region is a single chain Fv.

11. A recombinant immunotoxin polypeptide and pharmaceutically acceptable salts thereof according to claim 10 wherein the CD3-binding domain comprises a single chain Fv of UCHT-1.

12. A recombinant immunotoxin polypeptide and pharmaceutically acceptable salts thereof according to claim 1 comprising a single chain Fv of UCHT-1 fused to a PE mutant essentially deleted of its cell-binding domain.

13. A recombinant immunotoxin polypeptide and pharmaceutically acceptable salts thereof according to claim 12 wherein the PE mutant is PE38.

14. A recombinant immunotoxin polypeptide and pharmaceutically acceptable salts thereof according to claim 1 consisting essentially of the single chain Fv of an anti-human CD3 antibody fused via the carboxy terminus thereof to a PE mutant essentially deleted of its cell-binding domain.

15. A recombinant immunotoxin polypeptide and pharmaceutically acceptable salts thereof according to claim 14 having the formula V_{L}-L-V_{H}-C-PE mutant.

16. A recombinant immunotoxin polypeptide and pharmaceutically acceptable salts thereof according to claim 15 wherein V_{L} and V_{H} are derived from UCHT-1 and the PE mutant is PE38.

17. A recombinant immunotoxin polypeptide selected from polypeptides having residues 1-601, 2-601 and 3-601 of Sequence ID. NO: 1, homologs of said polypeptides which are at least 80% identical thereto, and their pharmaceutically acceptable salts.

18. A recombinant immunotoxin polypeptide according to claim 17 having residues 3-601 of SEQ. ID No:1 and its pharmaceutically acceptable salts.

19. A nucleic acid molecule encoding the recombinant immunotoxin polypeptide of claim 1.

20. A method of preparing a recombinant immunotoxin polypeptide of claim 1.

21. A method for treatment or prophylaxis of T-cell mediated disorders in a patient comprising administering to a patient in need thereof a therapeutically effective amount of a recombinant immunotoxin polypeptide or its pharmaceutically acceptable salt according to claim 1.

22. A method for treatment or prophylaxis of organ transplantation rejection in a transplant patient comprising administering to the patient a therapeutically effective amount of a recombinant immunotoxin polypeptide or its pharmaceutically acceptable salt according to claim 1.

23. A method for treatment or prophyaxis of autoimmune disease in a patient comprising administering to the patient a therapeutically effective amount of a recombinant immunotoxin polypeptide or its pharmaceutically acceptable salt according to claim 1.

24. An autologous therapy for treating or preventing a T-cell mediated disorder or condition in a patient, comprising:
(a) recruiting from the patient a cell population comprising CD3-bearing cells;
(b) treating the cell population with a recombinant immunotoxin polypeptide or its pharmaceutically acceptable salt according to claim 1 to at least partially deplete said cell population of CD3-bearing cells; and
(c) reinfusing the treated cell population into the patient.

25. A method for treatment or prophylaxis against graft versus host disease in patient to undergo a bone marrow transplant comprising:
(a) providing an inoculum comprising isolated bone marrow and/or stem cell-enriched peripheral blood cells of a suitable donor treated with a T-cell depleting effective amount of a recombinant immunotoxin polypeptide or its pharmaceutically acceptable salt according to claim 1; and
(b) transplanting the inoculum into the patient.

26. A method for the treatment or prophylaxis or treatment of transplant rejection in a patient to undergo a bone marrow transplant comprising:
(a) reducing the levels of viable CD3-bearing cell population in the patient;
(b) providing an inoculum comprising isolated bone marrow and/or stem cell-enriched peripheral blood cells of a suitable donor treated with a T-cell depleting effective amount of a recombinant immunotoxin polypeptide or its pharmaceutically acceptable salt according to claim 1; and
(c) introducing the inoculum into the patient, and thereafter optionally administering a recombinant immunotoxin polypeptide according to claim 1 to the patient to further deplete donor and patient T cells.

27. A method of conditioning a patient to be transplanted with cells, or a tissue or organ of a donor, the method comprising:
(a) depleting the CD3-bearing cell population in the patient;
(b) providing an inoculum comprising isolated bone marrow and/or stem-cell enriched peripheral blood cells of the donor treated with a T-cell depleting effective amount of a recombinant immunotoxin polypeptide or its pharmaceutically acceptable salt according to claim 1;
(c) introducing the inoculum into the patient; and
(d) transplanting the donor cells, tissue or organ into the patient.

28. A method according to claim 21 comprising co-administering the recombinant immunotoxin polypeptide or its pharmaceutically acceptable salt with at least one other pharmaceutical agent selected from cyclosporin A, rapamycin, 40-O-(2-hydroxy)ethyl rapamycin (RAD), FK-506, mycophenolic acid, mycophenolate mofetil (MMF), cyclophosphamide, azathioprene, leflunomide, mizoribine, a deoxyspergualine compound or derivative or analog, 2-amino-2-[2-(4-octylphenyl)ethyl]propane-1,3-diol, corticosteroids, anti-LFA-1 and anti-ICAM antibodies, and other antibodies that prevent co-stimulation of T cells.

29. A pharmaceutical composition comprising a recombinant immunotoxin polypeptide or its pharmaceutically acceptable salt according to claim 1 in a pharmaceutically acceptable carrier.
